(19) United States
(12) Patent Application Publication
(10) Pub. No.: US 2005/0048056 A1
(43) Pub. Date: Mar. 3, 2005

(54) Method for the preparation of molecules having antibody activity

(75) Inventors: Sa V. Ho, Ballwin, MO (US); Joseph K. McLaughlin, Ballwin, MO (US); Kristin Thomas, Wildwood, MO (US); Mark E. Gustafson, Saint Charles, MO (US); Amit Banerjee, Chesterfield, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia Corporation

(21) Appl. No.: 10/914,015

(22) Filed: Aug. 6, 2004

Related U.S. Application Data
(60) Provisional application No. 60/493,740, filed on Aug. 8, 2003.

(51) Int. Cl. A61K 39/395; C12P 21/04; C07K 16/24

(52) U.S. Cl. 424/145.1; 530/388.23; 435/70.21

(57) ABSTRACT

The present invention provides a process for the preparation of an antibody, wherein the method comprises: fermenting a cell mixture comprising the cells and a supernatant solution, wherein the cells are capable of expressing the light chain and the heavy chain; separating the cells from the supernatant solution to form a cell pellet; allowing the cell pellet to stand for a hold time; and extracting the cell pellet with an extracting solution; thereby producing the antibody.

### REFERENCE TO RELATED APPLICATIONS

The present application claims priority to U.S. Provisional Patent Application No. 60/493,740, filed Aug. 8, 2003.

### BACKGROUND OF THE INVENTION

1. Field of the Invention

This invention relates to the preparation of molecules having antibody activity.

2. Description of Related Art

Rheumatoid arthritis (herein, “RA”) is a crippling autoimmune condition which is characterized by synovial membrane proliferation and degradation of articular cartilage and subchondral bone. The condition is further characterized by increased levels of TNF-alpha in both the synovial fluid and peripheral blood of the patient.

Inflammatory Bowl Disease (herein, “IBD”) is a term which encompasses a variety of chronic inflammatory conditions of the large and small intestines. One such condition is Crohn's Disease, which can manifest symptoms such as inflammation of the bowel wall and can result in abscesses and fistulae (Goodman & Gilman's The Pharmacological Basis of Therapeutics, 10th Ed., J. G. Hardman and L. E. Limbird, eds., McGraw-Hill, New York, 2001, pp. 1047-8). TNF-alpha is expressed in the inflamed tissues associated with IBD.

RA and IBD have each been treated using methods designed to suppress or antagonize TNF-alpha. For example, infliximab is a monoclonal antibody to TNF-alpha which has been reported to be useful to treat either RA (id., p. 1475) or IBD (id., p. 1053). Another TNF-alpha monoclonal antibody which has been reported to be useful for the treatment of RA and IBD is etanercept.

A drawback to the therapeutic use of etanercept is that it requires a relatively frequent (every two weeks) subcutaneous injection. A drawback to the therapeutic use of infliximab is that its use requires intravenous administration at a physician's office. In addition, because both infliximab and etanercept are large proteins, their manufacture is difficult and expensive.

PCT Patent Application No. WO 01/94585 describes a molecule having antibody activity which is specific for human TNF-alpha and wherein the molecule comprises a TNF-alpha monoclonal antibody fragment (Fab′) and further comprising a methoxypolyethyleneglycolated (PEGylated) portion covalently attached to the Fab′ by a linker moiety. That application further describes methods of preparation and methods of using such a molecule. Such molecules are useful for the treatment or prevention of RA and of IBD.

The continued interest in the therapeutic uses of TNF-alpha antibodies indicates a strong need for discovery of new TNF-alpha molecules and for new methods to economically manufacture them.

### SUMMARY OF THE INVENTION

Among the several embodiments of the present invention may be noted the provision of a process for the preparation of an antibody or an antibody fragment or a dimer or adduct thereof wherein the method comprises: fermenting a cell mixture comprising the cells and a supernatant solution, wherein the cells are capable of expressing the light chain and the heavy chain; separating the cells from the supernatant solution to form a cell pellet; allowing the cell pellet to stand for a hold time; and extracting the cell pellet with an extracting solution; thereby producing the antibody or antibody fragment or a dimer or an adduct thereof.

Further scope of the applicability of the present invention will become apparent from the detailed description provided below. However, it should be understood that the following detailed description and examples, while indicating embodiments of the invention, are given by way of illustration only since various changes and modifications within the spirit and scope of the invention will become apparent to those skilled in the art from this detailed description.

### BRIEF DESCRIPTION OF THE DRAWINGS

FIG. 1. Comparison of framework regions of light chain of antibody hTNF-40 and human group 1 consensus sequences.

FIG. 2. Comparison of framework regions of heavy chain of antibody hTNF-40 and human group 1 and group 3 consensus sequences.

FIG. 3. Sequence of CDRs of hTNF-40.

FIG. 4. Schematic of vector pMR 15.1.

FIG. 5. Schematic of vector pMR14.

FIG. 6. The nucleotide and predicted amino acid sequence of the murine hTNF40VI (SEQ ID NO: 99).

FIG. 7. The nucleotide and predicted amino acid sequence of the murine hTNF40Vh (SEQ ID NO: 100).

FIG. 8. The nucleotide and predicted amino acid sequence of hTNF46-gLI (SEQ ID NO: 8).

FIG. 9. The nucleotide and predicted amino acid sequence of hTNF40-gL2 (SEQ ID NO:9).

FIG. 10. The nucleotide and predicted amino acid sequence of ghlhTNF40.4 (SEQ ID NO: 10).

FIG. 11. The nucleotide and predicted amino acid sequence of gh3hTNF40.4 (SEQ ID NO: 11).

FIG. 12. Schematic of vector CTIL5-gL6.

FIG. 13. The structure of a compound called CDP-870 comprising a modified Fab′ fragment derived from antibody hTNF40 covalently linked via a cysteine residue to a lysyl-maleimide linker wherein each amino group on the lysyl residue has covalently attached to it a methoxy PEG residue wherein n is about 420.

FIG. 14. Schematic of vector pTTQ9.

FIG. 15. The sequence of the OmpA oligonucleotide adapter (SEQ ID NO: 10 1).

FIG. 16. Schematic of vector PACYC 184.

FIG. 17. Schematic of vector pTTO-1.

FIG. 18. Schematic of vector pTTO-2.

FIG. 19. Schematic of vector pDNAbEng-Gl.

FIG. 20. The oligonucleotide cassettes encoding different intergenic sequences for E. coli modified Fab′ expression (SEQ ID NOS: 102 to 105).

FIG. 21. Graph of the periplasmic modified Fab′ accumulation of IGS variants.

FIG. 22. Schematic of vector pTTO(CDP-870).

FIG. 23. Flow chart for the fermentation step in the synthesis of CDP-870 Fab′-PEG.

FIG. 24. Flow chart for the primary separation step in the synthesis of CDP-870 Fab′-PEG.

FIG. 25. Effect of pellet hold time on Fab′ recovery.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

a. Definitions

The following definitions are provided in order to aid the reader in understanding the detailed description of the present invention:

“AEX” as used herein means anion exchange chromatography.

The term “antibody” as used herein means an antigen-binding protein and includes complete antibody molecules, antibody fragments, chimeric antibodies, chimeric antibody fragments, and chemically modified antibodies or antibody fragments (e.g., PEGylated).

“CDP-870 Fab′-PEG” as used herein is used interchangeably with the term “CDP-870” and means the CDP-870 molecule described in PCT Patent Application No. WO 01/94585.

“CDR” means complementary determining region.

“CEX” as used herein means cation exchange chromatography.

“EBA” as used herein means expanded bed adsorption or expanded bed adsorption chromatography.

“EDTA” as used herein means ethylenediaminetetraacetic acid.

“Fab′” as used herein means an antibody fragment having a heavy chain and a light chain. When used in the context of CDP-870, “Fab′” means a TNF-alpha antibody wherein the heavy chain of the antibody 5′ part has the sequence given as SEQ ID NO: 115 and the light chain has the sequence given in SEQ ID NO: 113. In the context of CDP-870, this term is used interchangeably with the term “CDP-870 Fab′.”

“F(ab′)₂” means a dimer of Fab′.

“Fab′ Species” as used herein means any or all of Fab′, F(ab′)₂, Fab′ adducts, and any other species that can be reduced to form Fab′

“HCP” as used herein means host cell proteins.

“HIC” as used herein means hydrophobic interaction chromatography.

“NaOAc” as used herein means sodium acetate.

“OD₆₀₀” as used herein means optical density at 600 nanometers.

“PEG” as used herein means polyethylene glycol in which one terminal hydroxyl group is optionally capped with a methyl (i.e., methoxy(polyethyleneglycol)).

“PEGylated” as used herein means having a chemical moiety containing PEG attached thereto.

“Tris” as used herein means tris(hydroxymethyl)-aminomethane.

b. Process Details

In accordance with the present invention, a process has been discovered for economically producing an antibody (including Fab′ and F(ab′) ₂). In one embodiment, the present invention provides a method for the production of a molecule having antibody activity which is specific for human TNF-alpha. For example, the present invention provides methods for the production of antibody molecules wherein the molecule comprises a TNF-alpha monoclonal antibody fragment (Fab′) and further comprising a methoxypolyethyleneglycolated (PEGylated) portion covalently attached to the Fab′ by a linker moiety. Particularly, the present application describes a method for the preparation of such a molecule known as CDP-870. CDP-870 is described in PCT Patent Application No. WO 01/94585 (U.S. patent application No. 09/875,221, herein incorporated by reference).

In one embodiment, the present invention provides a method of preparing an antibody molecule or antibody molecule fragment having specificity for TNF-alpha, wherein the molecule or fragment comprises a heavy chain having a variable domain comprising a CDR having the sequence given as HI in FIG. 3 (SEQ ID NO: 1) for CDRH1, as H2′ in FIG. 3 (SEQ ID NO:2) or as H2 in FIG. 3 (SEQ ID NO:7) for CDRH2 or as H3 in FIG. 3 (SEQ ID NO:3) for CDRH3. The antibody molecule or fragment comprises at least one CDR selected from H 1, H2′ or H2 and H3 (SEQ ID NO: 1; SEQ ID NO:2 or SEQ ID NO:7 and SEQ ID NO:3) for the heavy chain variable domain. In one embodiment, the antibody molecule comprises at least two or all three CDRs in the heavy chain variable domain.

The present invention further provides a method of preparing an antibody molecule having specificity for human TNF-alpha, wherein the antibody molecule comprises a light chain having a variable domain which comprises a CDR having the sequence given as L1 in FIG. 3 (SEQ ID NO:4) for CDRL1, L2 in FIG. 3 (SEQ ID NO:5) for CDRL2 or L3 in FIG. 3 (SEQ ID NO:6) for CDRL3. This antibody molecule or fragment comprises at least one CDR selected from L1, L2 and L3 (SEQ ID NOA to SEQ ID NO:6) for the light chain variable domain. In one embodiment, the antibody molecule comprises at least two or all three CDRs in the light chain variable domain.

In one embodiment, the antibody molecules or fragments have a complementary light chain or a complementary heavy chain, respectively.

In one embodiment, the antibody molecule or fragments comprise a heavy chain wherein the variable domain comprises a CDR having the sequence given as H1 in Figure (SEQ ID NO: 1) for CDRH1, as H2′ or H2 in FIG. 3 (SEQ ID NO:2 or SEQ ID NO:7) for CDRH2 or as H3 in FIG. 3 (SEQ ID NO:3) for CDRH3 and a light chain wherein the variable domain comprises a CDR having the sequence given as LI in FIG. 3 (SEQ ID NO:4) for CDRL1, as L2 in FIG. 3 (SEQ ID NO:5) for CDRL2 or as L3 in FIG. 3 (SEQ ID NO:6) for CDRL3.

The CDRs given in SEQ IDS NOS: 1 and 3 to 7 and in FIG. 3 referred to above are derived from a mouse monoclonal antibody hTNF40. However, SEQ ID NO:2 comprises a hybrid CDR. The hybrid CDR comprises part of heavy chain CDR2 from mouse monoclonal antibody hTNF40 (SEQ ID NO:7) and part of heavy chain CDR2 from a human group 3 germline V region sequence.

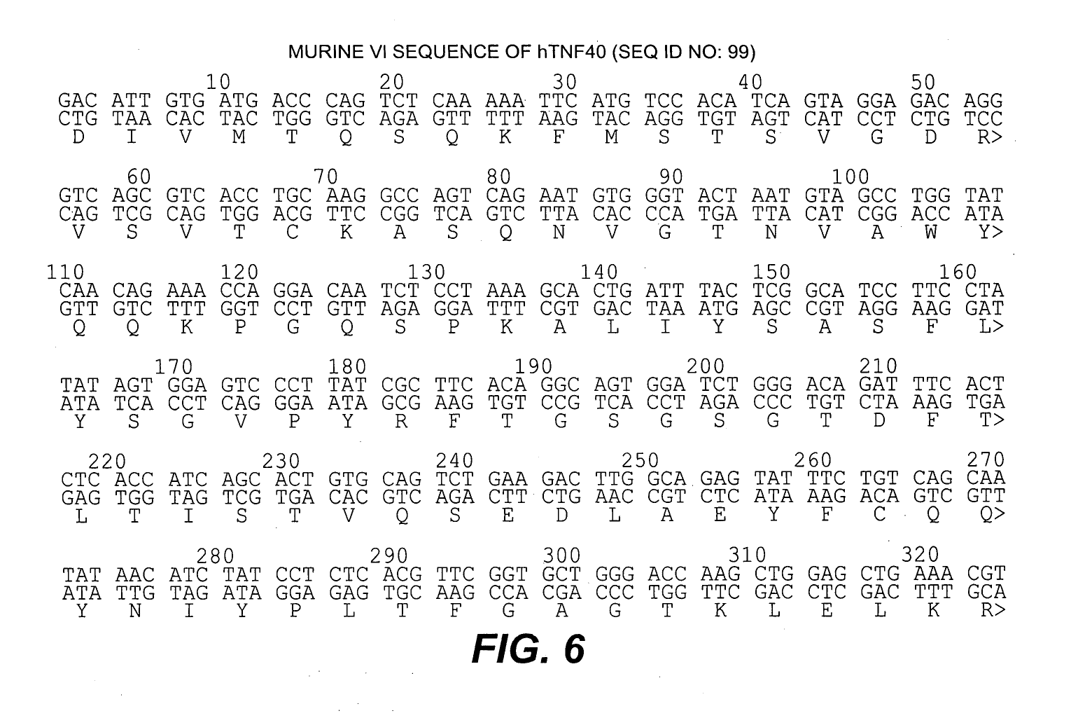

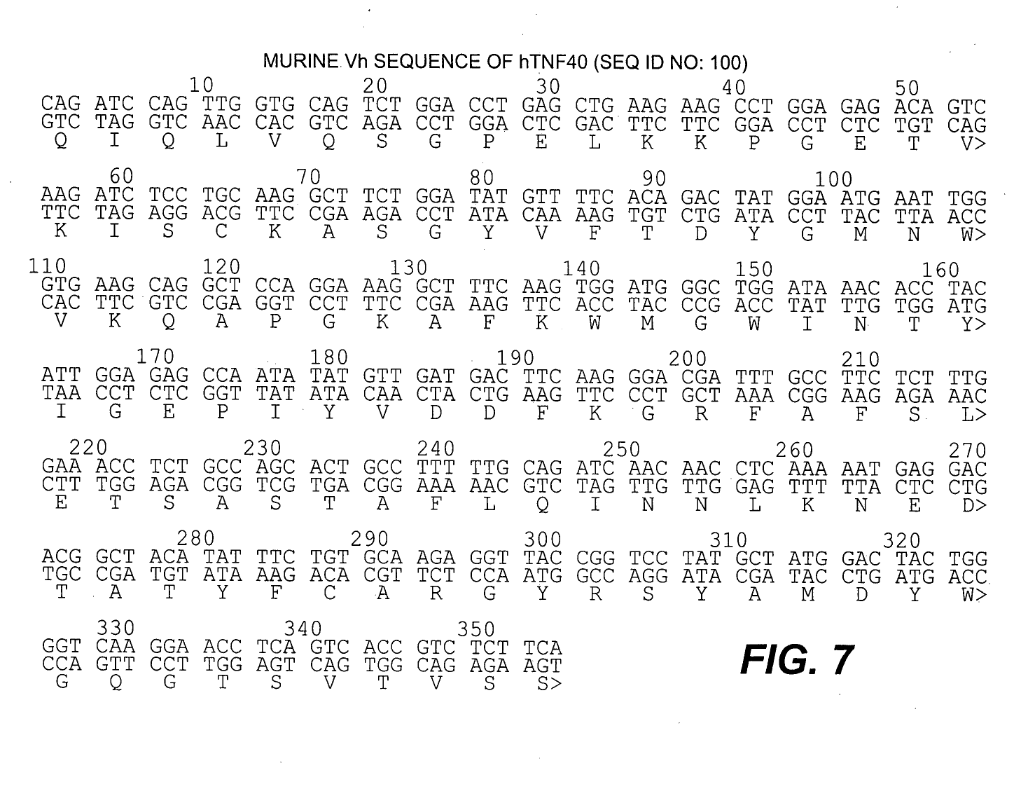

The complete sequences of the variable domains of the mouse hTNF40 antibody 10 are shown in FIGS. 6 (light chain) (SEQ ID NO:99) and FIG. 7 (heavy chain) (SEQ ID NO: 100). This mouse antibody is referred to below as “the donor antibody”.

In an alternative embodiment, the present invention provides a method of preparing a mouse monoclonal antibody hTNF40 having the light and heavy chain variable domain sequences shown in FIG. 6 (SEQ ID NO:99) and FIG. 7 (SEQ ID NO: 100), respectively. The light chain constant region of hTNF40 is kappa and the heavy chain constant region is IgG2a.

In another embodiment, the antibody is a chimeric mouse/human antibody molecule or fragment, referred to herein as the chimeric hTNF40 antibody molecule. The chimeric antibody molecule comprises the variable domains of the mouse monoclonal antibody hTNF40 (SEQ ID NOS:99 and 100) and human constant domains. In another embodiment, the chimeric hTNF40 antibody molecule comprises the human C kappa domain (Hieter et al., Cell, 22, 197-207, 1980; Genebank accession number J00241) in the light chain and the human gamma 4 domains (Flanagan et al., Nature, 300, 709-713, 1982) in the heavy chain.

In another embodiment, the antibody is a CDR-grafted antibody molecule or fragment. The term “a CDR-grafted antibody molecule” as used herein refers to an antibody molecule wherein the heavy and/or light chain contains one or more CDRs (including, if desired, a hybrid CDR) from the donor antibody (e.g. a murine monoclonal antibody) grafted into a heavy and/or light chain variable region framework of an acceptor antibody (e.g. a human antibody).

In one embodiment, such a CDR-grafted antibody has a variable domain comprising human acceptor framework regions as well as one or more of the donor CDRs referred to above.

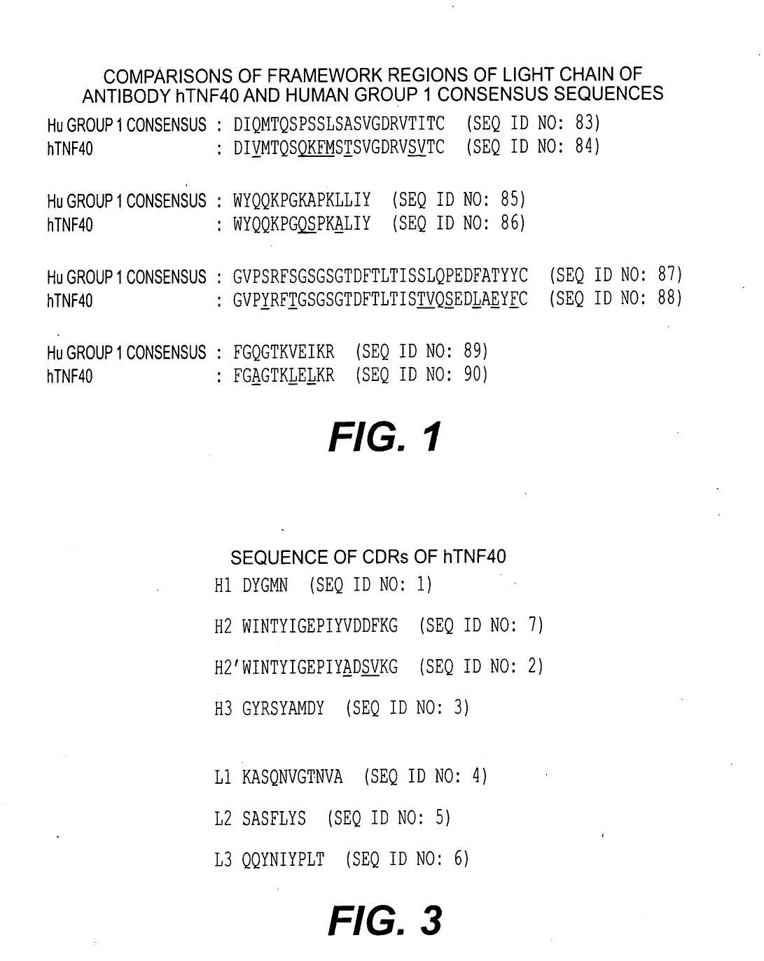

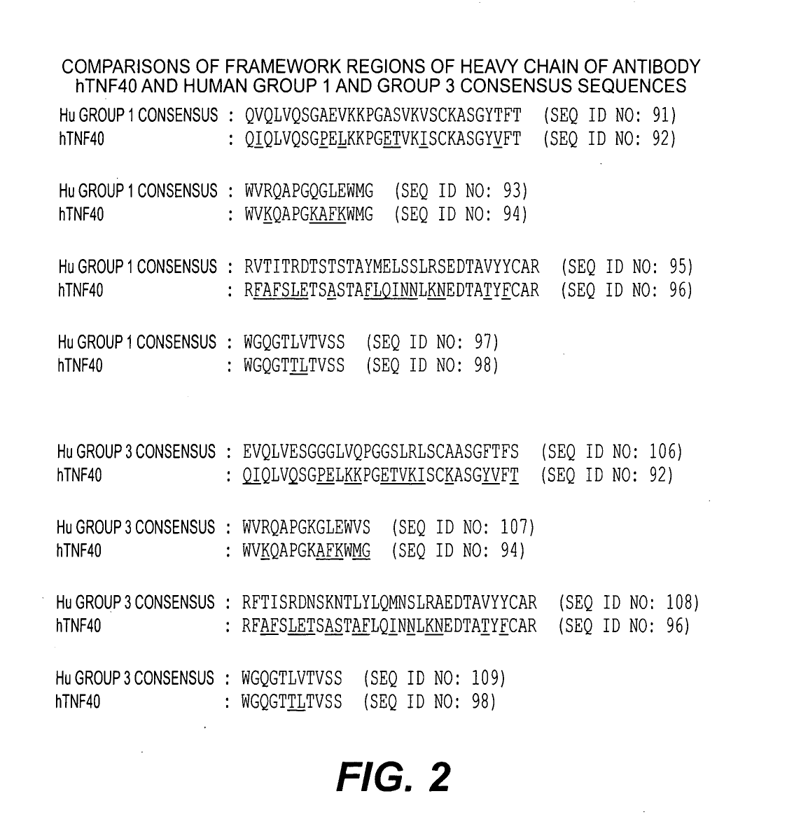

When the CDRs are grafted, any appropriate acceptor variable region framework sequence may be used having regard to the class or type of the donor antibody from which the CDRs are derived, including mouse, primate and human framework regions. Examples of human frameworks which can be used in the present invention are KOL, NEWM, REI, EU, TUR, TEI, LAY and POM. For example, KOL and NEWM can be used for the heavy chain, REI can be used for the light chain and EU, LAY and POM can be used for both the heavy chain and the light chain. Useful framework regions for the light chain include the human group I framework regions shown in FIG. 1 (SEQ ID NOS:83, 85, 87 and 89). Useful framework regions for the heavy chain include the human group 1 and group 3 framework regions shown in FIG. 2 (SEQ ID NOS:91, 93, 95 and 97 and SEQ ID NOS: 106, 107, 108 and 109), respectively.

In a CDR-grafted antibody, it is possible to use an acceptor antibody having chains which are homologous to the chains of the donor antibody. The acceptor heavy and light chains do not necessarily need to be derived from the same antibody and may, if desired, comprise composite chains having framework regions derived from different chains.

In the CDR-grafted antibody molecule of the present invention, if the acceptor heavy chain has human group I framework regions (shown in FIG. 2) (SEQ ID NOS:91, 93, 95 and 97), then the acceptor framework regions of the heavy chain comprise, in addition to one or more donor CDRs, donor residues at positions 28, 69 and 71.

Alternatively, if the acceptor heavy chain has group I framework regions, then the acceptor framework regions of the heavy chain comprise, in addition to one or more donor CDRs, donor residues at positions 28, 38, 46, 67, 69 and 71.

Alternatively, in the CDR-grafted antibody molecule, if the acceptor heavy chain has human group 3 framework regions (shown in FIG. 2) (SEQ ID NOS:106, 107, 108 and 109), then the acceptor framework regions of the heavy chain comprise, in addition to one or more donor CDRs, donor residues at positions 27, 28, 30, 48, 49, 69, 71, 73, 76 and 78.

Alternatively, in a CDR-grafted antibody molecule, if the acceptor light chain has human group I framework regions (shown in FIG. 1) (SEQ ID NOS:83, 85, 87 and 89) then the acceptor framework regions of the light chain comprise donor residues at positions 46 and 60.

Donor residues are residues from the donor antibody, i.e. the antibody from which the CDRs were originally derived.

The antibody molecule (whether a TNF-alpha antibody or another antibody) made by the present invention may comprise: a complete antibody molecule, having full length heavy and light chains; a fragment thereof, such as a Fab, modified Fab, Fab′, F(ab′)2 or Fv fragment; a light chain or heavy chain monomer or dimer; a single chain antibody, e.g. a single chain Fv in which the heavy and light chain variable domains are joined by a peptide linker. Similarly, the heavy and light chain variable regions may be combined with other antibody domains as appropriate.

In one embodiment the antibody molecule made by the present method is a Fab′ (i.e., an antibody fragment).

For example the Fab′ can comprise a heavy chain having the sequence given as SEQ ID NO: 111 and a light chain having the sequence given as SEQ ID NO: 113. The amino acid sequences given in SEQ ID NO: 111 and SEQ ID NO: 113 can be encoded by the nucleotide sequences given in SEQ ID NO: 110 and SEQ ID NO: 112, respectively.

Alternatively, the antibody molecule made by the present invention can comprise a modified Fab′ fragment wherein the modification is the addition to the C-terminal end of its heavy chain one or more amino acids to allow the attachment of an effector or reporter molecule. In one embodiment, the additional amino acids form a modified hinge region containing one or two cysteine residue to which the effector or reporter molecule may be attached. In one embodiment, such a modified Fab′ fragment may have has a heavy chain having the sequence given as SEQ ID NO: 115 and the light chain having the sequence given as SEQ ID NO: 113. The amino acid sequence given in SEQ ID NO: 115 can be encoded by the nucleotide sequence given in SEQ ID NO: 114.

A useful effector group is a polymer molecule, which may be attached to the modified Fab′ fragment to increase its half-life in vivo.

The polymer molecule may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Particular optional substituents which may be present on the above-mentioned synthetic polymers include one or more hydroxy, methyl or methoxy groups. Particular examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof. Particular naturally occurring polymers include amylose, dextran, glycogen or derivatives thereof. Alternatively, a monomer such as lactose can be used. “Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

The size of the polymer may be varied as desired, but will generally be in an average molecular weight range from 500 Da to 50,000 Da, for example from 5000 to 40,000 Da and in another embodiment from 25,000 to 40,000 Da. The polymer size may in particular be selected on the basis of the intended use of the product. Thus, for example, where the product is intended to leave the circulation and penetrate tissue, for example for use in the treatment of a tumor, it may be advantageous to use a small molecular weight polymer, for example with a molecular weight of around 5000 Da. For applications where the product remains in the circulation, it may be advantageous to use a higher molecular weight polymer, for example having a molecular weight in the range from 25,000 Da to 40,000 Da. Particularly useful polymers include a polyalkylene polymer, such as a poly(ethyleneglycol) or, especially, a methoxypoly(ethyleneglycol) or a derivative thereof, and especially with a molecular weight in the range from about 25,000 Da to about 40,000 Da. Each polymer molecule attached to the modified antibody fragment may be covalently linked to the sulphur atom of a cysteine residue located in the fragment. The covalent linkage will generally be a disulphide bond or, in particular, a sulphur-carbon bond.

Where desired, the antibody fragment may have one or more effector or reporter molecules attached to it. The effector or reporter molecules may be attached to the antibody fragment through any available amino acid side-chain or terminal amino acid functional group located in the fragment, for example any free amino, imino, hydroxyl or carboxyl group.

An activated polymer may be used as the starting material in the preparation of polymer-modified antibody fragments as described above. The activated polymer may be any polymer containing a thiol reactive group such as an cc-halocarboxylic acid or ester, e.g. iodoacetamide, an imide, e.g. maleimide, a vinyl sulphone or a disulphide. Such starting materials may be obtained commercially (for example from Nektar Therapeutics, Huntsville, Ala., USA) or may be prepared from commercially available starting materials using conventional chemical procedures.

Where it is desired to obtain an antibody fragment linked to an effector or reporter molecule, this may be prepared by standard chemical or recombinant DNA procedures in which the antibody fragment is linked either directly or via a coupling agent to the effector or reporter molecule either before or after reaction with the activated polymer as appropriate. Particular chemical procedures include for example, those described in each of the following individual references:
WO 93/62331.
WO 92/22583.
WO 90/00195.
WO 89/01476.

Alternatively, where the effector or reporter molecule is a protein or polypeptide the linkage may be achieved using recombinant DNA procedures, for example as described in WO 86/01533. Another example is described in EP-A 0392745.

In one embodiment, the modified Fab′ fragment of the present invention is PEGylated (i.e. has PEG (poly(ethyleneglycol)) or methyl-capped (poly(ethyleneglycol)) covalently attached thereto). A method for covalently attaching PEG is described in EP-A-0948544. In one embodiment the antibody molecule of the present invention is a PEGylated modified Fab′ fragment as shown in FIG. 13. In FIG. 13, the modified Fab′ fragment has a maleimide group covalently linked to a single thiol group in a modified hinge region. A lysine residue is covalently linked to the maleimide group. To each of the amine groups on the lysine residue is attached a methoxypoly(ethyleneglycol) polymer having a molecular weight of approximately 20,000 Da. The total molecular weight of the entire effector molecule is therefore approximately 40,000 Da.

In one embodiment, in the compound shown in FIG. 13, the heavy chain of the antibody 5′ part has the sequence given as SEQ ID NO: 115 and the light chain has the sequence given in SEQ ID NO: 113. This compound is referred to herein as CDP-870.

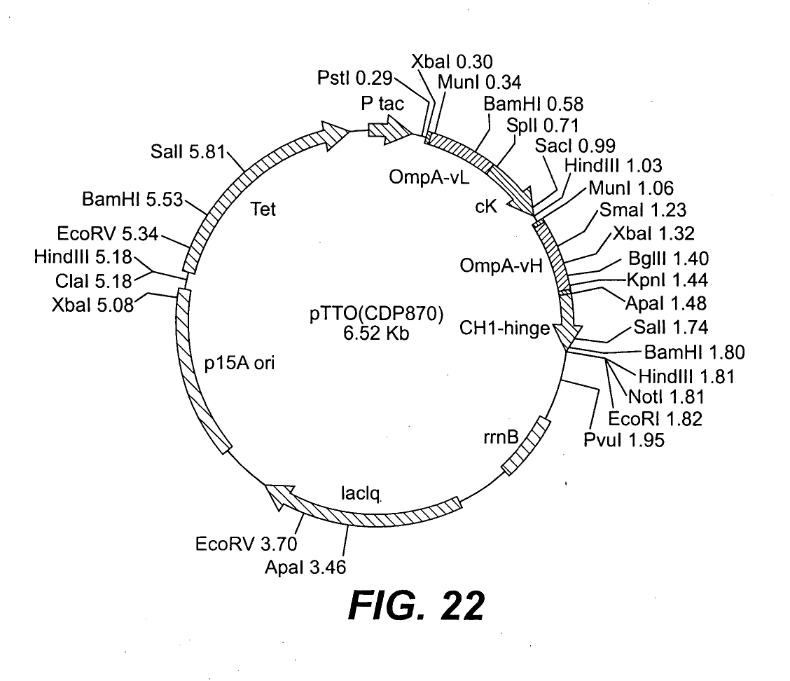

In another embodiment, the present method provides an E. coli expression vector comprising a DNA sequence of the present invention. In another embodiment the expression vector is PTTO(CDP-870) as shown schematically in FIG. 22.

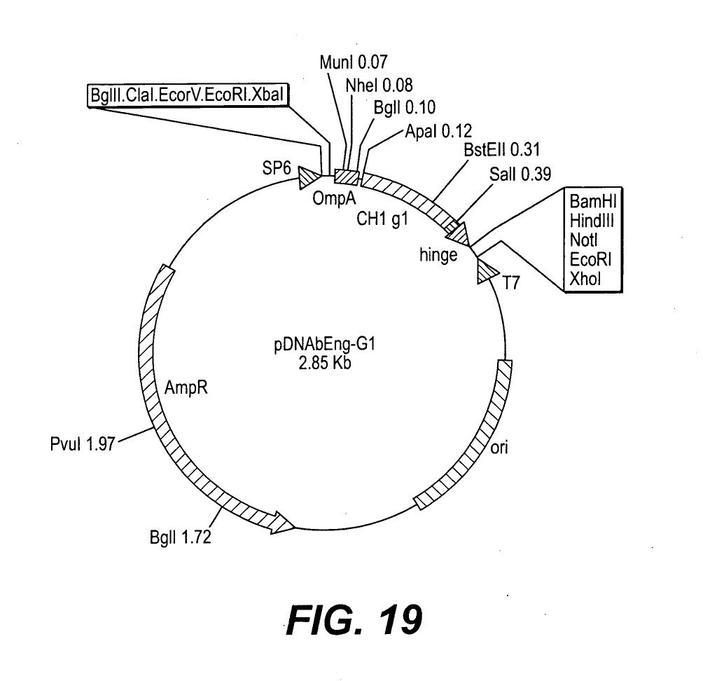

The present invention also provides vector pDNAbEng-Gl as shown in FIG. 19.

DNA sequences coding for part or all of the antibody heavy and light chains may be synthesized as desired from the determined DNA sequences or on the basis of the corresponding amino acid sequences.

Standard techniques of molecular biology may be used to prepare DNA sequences coding for the antibody molecule made by the present invention. Desired DNA sequences may be synthesised completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecule made by the present invention. Bacterial, for example E. coli, and other microbial systems may be used, in part, for expression of antibody fragments such as Fab′ and F(ab′)2 fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, myeloma. or hybridoma cells.

The present invention also provides a process for the production of an antibody molecule wherein the process comprises culturing a host cell comprising a vector of the present invention, wherein the culturing is performed under conditions suitable for leading to expression of protein from DNA encoding the antibody molecule of the present invention, and isolating the antibody molecule.

The process for the production of the antibody molecule made by the present invention can comprise culturing E. coli bacteria wherein the bacteria comprise an E. coli expression vector comprising the DNA sequence of the present invention, and wherein the culturing is performed under conditions suitable for leading to expression of protein from the DNA sequence and isolating the antibody molecule. The antibody molecule may be secreted from the cell or targeted to the periplasm by suitable signal sequences. Alternatively, the antibody molecules may accumulate within the cell's cytoplasm. In one embodiment the antibody molecule is targeted to the periplasm. Depending on the antibody molecule being produced and the process used, it is desirable to allow the antibody molecules to refold and adopt a functional conformation. Procedures for allowing antibody molecules to refold are well known to those skilled in the art.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

The antibody molecules made by the present invention can be prepared, for example, by the methods exemplified below. In these methods, CDP-870 Fab′-PEG is used as an example, but other antibody molecules can be prepared by similar methods. CDP-870 Fab′-PEG is prepared using a recombinant E. coli expression system, W3110 pTTOCDP-870. The system utilizes the lacIq gene encoding the lacI repressor to control the activity of the tac promoter driving transcription of the antibody genes. A DNA sequence encoding the OmpA leader sequence is placed directly upstream of the structural genes for both antibody chains. This polypetide sequence directs translocation of the polypeptides to the E. coli periplasm and is cleaved from the N-terminal of the antibody chains during this process.

The recombinant E. coli can be grown on a medium containing salts, trace metals, and a source of carbon and metabolic energy, and then induced to express CDP-870 Fab′ and other Fab′ species. The source of carbon and metabolic energy can vary widely. For example the source of carbon and metabolic energy can be a carbohydrate. The carbohydrate can be, for example, glycerol or a saccharide. When the carbohydrate is a saccharide, it can be a monosaccharide or it can be a polysaccharide (where a polysaccharide contains two or more saccharide subunits). When the carbohydrate is a saccharide, it is advantageously a monosaccharide such as glucose. In one useful embodiment the source of carbon and metabolic energy is glycerol. When the optical density (OD₆₀₀) of the growth medium reaches approximately 40-50 (conveniently about 45), a protein production inducer (herein, “inducer”) can be added to the fermentation. The inducer can vary widely in nature. For example, the inducer can include lactose or IPTG. The start of induction is defined as the point at which the source of carbon and metabolic energy (e.g., glycerol) is depleted from the medium. Inducer (e.g., lactose) levels can be maintained and CDP-870 Fab′ may be allowed to accumulate for a period of time. The period of time during which the CDP-870 Fab′ accumulates can be varied depending on the desired results. It is convenient to allow the CDP-870 Fab′ to accumulate for about 1 to about 50 hours, alternatively for about 10 to about 40 hours, alternatively for about 20 to about 35 hours, alternatively for about 30 hours. After the accumulation time, the fermentation broth can be cooled and the cells harvested by an appropriate method. Useful cell harvest methods include, but are not limited to, centrifugation or filtration. The periplasmic Fab′ can be extracted by resuspension of the cell slurry in extraction buffer and incubation. Incubation can be carried out at a variety of temperatures, conveniently at a temperature less than about 65° C., alternatively about 50 to about 620° C., alternatively about 55 to about 620° C., alternatively at about 600° C. Extraction time can vary widely. It is useful to extract, for example, for 0.1 to about 48 hours, alternatively 0.5 to about 36 hours, alternatively about 1 to about 24 hours, alternatively 5 to about 15 hours, alternatively about 10 hours. After the extraction, the suspension can be cooled and/or pH adjusted, if desired. If the pH is adjusted, a variety of acids or bases can be used. Commonly it is necessary to lower the pH. For this purpose, a variety of acids can be used, for example organic acids or mineral acids. It is convenient to use an organic acid for this purpose. Useful organic acids include carbonic acid or a carboxylic acid. When a carboxylic acid is used, it can conveniently be a C₁ to about C₁₀ carboxylic acid, alternatively a C₁ to about C₅ carboxylic acid, alternatively a C₁ to about C₃ carboxylic acid, and alternatively acetic acid. If the pH is adjusted, it is convenient to adjust it to about 4 to about 5, alternatively about 4.5. If any cell debris, precipitated proteins, or aggregates are present, they can be conveniently removed by a variety of methods. Convenient methods for the removal of cell debris, precipitated proteins, or aggregates include chromatographic methods, for example through the use of an ion exchange chromatographic method. Useful ion exchange chromatographic methods include anionic exchange chromatography and cationic exchange chromatography. Conveniently, one can use Cation Exchange Expanded Bed Adsorption Chromatography for separating cell debris, precipitated proteins, or aggregates. The CDP-870 Fab′ can be further purified by one or more additional chromatography steps. Such additional chromatography steps include anion exchange chromatography (herein, “AEX”), cation exchange chromatography (herein, “CEX”), hydrophobic interaction chromatography (herein, “HIX”), and others. It is convenient to use two or more column chromatography steps, such as Anion Exchange Chromatography (AEX) and Hydrophobic Interaction Chromatography (HIC). Ultrafiltration/diafiltration (herein, “UF/DF”) can be used before or after the additional chromatography step(s) for further purification of the CDP-870 Fab′. For example, UF/DF can be used before an AEX step and after an HIC step (where AEX and HIC are used sequentially) to adjust CDP-870 Fab′ concentration or to exchange buffers.

The CDP-870 Fab′ can be treated with a reductant to activate the single Fab′ hinge thiol. Such reduction can conveniently be performed after a purification procedure as described above.

c. Detailed Preparative Methods

The starting materials for use in the methods of preparation of the invention are known or can be prepared by conventional methods known to a skilled person or in an analogous manner to processes described in the art.

Generally, the process methods of the present invention are exemplified as follows.

### EXAMPLE 1. CDP-870 Fab′

Fermentation

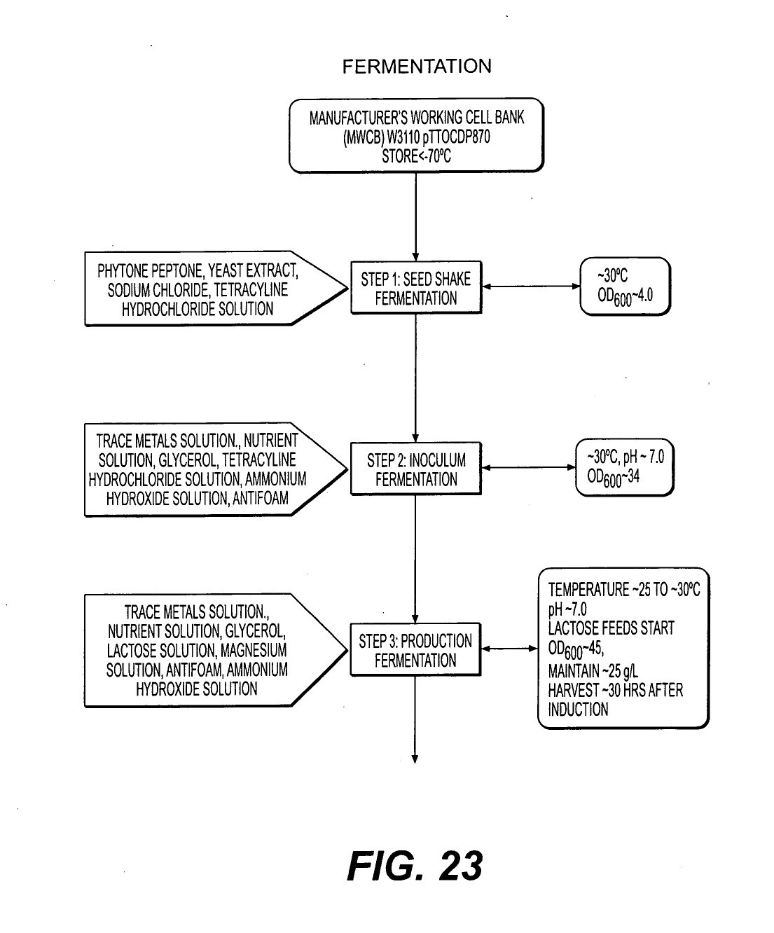

Conveniently, the fermentation stage can comprise steps including: Seed Shake Fermentation, Inoculum Fermentation, and Production Fermentation. An example of the Fermentation Stage is shown schematically in FIG. 23.

A purpose of Seed Shake Fermentation (Step 1) is to propagate cells. This step can begin when shake flasks containing Phytone, Yeast Extract, and sodium chloride, with Tetracycline Solution (about 5 micrograms/mL of tetracycline) added as a selective agent, are inoculated with a seed vial (i.e., a vial containing approximately 1 ml of cell culture to inoculate the seed flask to start the fermentation process). The flasks can be incubated at a temperature of about 25 to about 35° C., alternatively at about 300° C., with agitation until OD₆₀₀ of the medium reaches about 2 to about 5, for example about 4. A total of approximately three liter of seed shake culture is pooled from five flasks and transferred to the inoculum fermenter.

A purpose of Inoculum Fermentation (Step 2) is to further propagate cells to a concentration that is sufficient for inoculating the production fermentor. This step can begin when Nutrient Solution (defined below) is inoculated with the seed shake culture. Tetracycline Solution is added to the Nutrient Solution as a selective agent, Antifoam (e.g., PPG 2000) can be added to prevent foaming, and Ammonium Hydroxide Solution can be added to control pH. The culture parameters of temperature, pH, agitation, backpressure, and aeration are controlled. For examples of useful culture parameters, see FIG. 23. The culture is harvested when the OD₆₀₀ reaches about 30 to about 40, alternatively about 34.

Nutrient Solution:

| Component | Concentration | |
|---|---|---|
| Citric acid | 0.005 | Kg/L |
| Magnesium sulfate.7H₂O | 0.001 | Kg/L |
| Sodium dihydrogen phosphate.2H₂O | 0.005 | Kg/L |
| Ammonium sulfate | 0.005 | Kg/L |
| Potassium chloride | 0.004 | Kg/L |
| Calcium chloride.2H₂O | 0.291 | g/L |
| Glycerol | 0.097 | Kg/L |
| Zinc sulfate.7H₂O | 0.020 | g/L |
| Manganese sulfate.1H₂O | 0.020 | g/L |
| Copper sulfate.5H₂O | 0.008 | g/L |
| Cobalt sulfate.7H₂O | 0.004 | g/L |
| Iron (III) chloride hexahydrate | 0.100 | g/L |
| Boric acid | 0.0003 | g/L |
| Sodium molybdate.2H₂O | 0.0002 | g/L |
| Foam inhibitor PPG | 0.250 | mL/L |

A purpose of Production Fermentation (Step 3) is to propagate cells to a desired concentration where they are induced to synthesize CDP-870 Fab′. This step begins when approximately 300 L of the seeding bioreactor is used to inoculate Nutrient Solution (see Step 2) in the production bioreactor. The culture parameters of temperature, pH, agitation, backpressure, and aeration are controlled. For examples of useful culture parameters for this step, see FIG. 23. Antifoam (e.g., PPG 2000) can be added to control foaming, Magnesium Solution and Lactose Solution are added to supplement the medium. Ammonium Hydroxide Solution and sulfuric acid (if necessary) are added to control pH. The culture is stopped (e.g., the production bioreactor or fermenter is harvested) when the time post-induction is about 10 to about 60 hours, alternatively about 20 to 40 hours, alternatively about 30 hours.

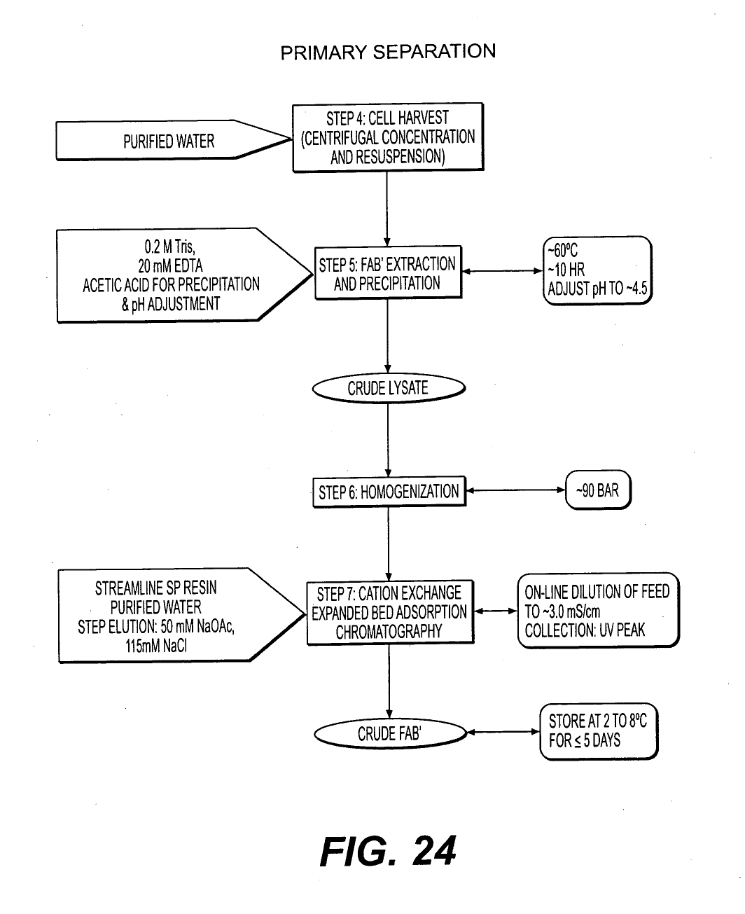

Primary Separation of CDP-870 Fab′ can include the following four steps: Cell Harvest, Fab′ Extraction and Precipitation, Homogenization, and Chromatographic Separation. An example of the Primary Separation stage is shown schematically in FIG. 24.

A purpose of Cell Harvest (Step 4) is to separate cells containing CDP-870 Fab′ from the fermentation medium. After fermentation, the cells are separated for example by filtration or centrifugation. The supernatant is discarded. The wet cells can be collected and diluted with purified water to -50% of the original harvest broth volume. Alternatively, it is now reported that holding the cell paste (either as an isolated pellet, or in the presence of supernatant or other liquid) for a period of time prior to the extraction step will result in an increase in the amount of Fab′ extracted into solution relative to the situation in which the cell paste is extracted immediately after Cell Harvest.

The example in Table 1, discussed in more detail below, shows over 30% increase in extraction yield was achieved by holding the cell paste at −20° C. for about 30 hrs or longer prior to carrying out the typical extraction process described above.

A purpose of Fab′ Extraction and Precipitation (Step 5) is to release CDP-870 Fab′ from the cells. An extracting solution (e.g., a buffer solution such as 0.2 M Tris and 20 mM EDTA) is added to the harvested and diluted cells and the solution is heated to about 50 to about 65° C., alternatively about 60° C. for approximately 2 to about 30 hours, alternatively about 5 to about 20 hours, alternatively about 10 hours. The solution can then be cooled, e.g., to about 10 to about 50° C., alternatively about 10 to about 30° C., alternatively about 15 to about 20° C., alternatively about 19 to about 20° C. The pH during this process, if desired, can be higher than about 3, alternatively higher than about 4.5, alternatively higher than about 7, and alternatively higher than about 8. A convenient buffer system to use for pH adjustment is acetate buffer. If desired, the extraction can be performed in the presence of a chaotropic agent (e.g., urea) or a detergent.

A purpose of Homogenization (Step 6) is to prepare the process stream for the following chromatography step. The extraction solution is homogenized at low pressure to create a homogeneous slurry.

A purpose of Cation Exchange Expanded Bed Adsorption Chromatography (Step 7) is to separate CDP-870 Fab′ from the cells and debris and to remove host cell proteins (herein, “HCP”) and endotoxin. The homogenized crude extract can be, if desired, diluted online with purified water (e.g., approximately 3.5 fold) during the load to a chromatography column. A variety of chromatography columns can be sued, for example an expanded bed chromatography column or a fixed bed chromatography column. It is convenient to use an Expanded Bed Adsorption (herein, “EBA”) chromatography column in expanded bed mode. A convenient resin is a cation exchanger, such as an agarose-derived resin. A useful cation exchanger is Streamline SP (Amersham). After a washing step with equilibration buffer in both the expanded and fixed bed modes, bound Fab′ is eluted with a 50 mM NaOAc, 115 mM NaCl buffer in fixed bed mode. The collected product can be either processed immediately, or held for a period of time (e.g., hours or days). When the collected product is held, it is convenient to hold it at sub-ambient temperature, for example at about 0 to about 150° C., alternatively about 2 to about 80° C. Also, EBA pool lots may be combined for further downstream processing. RP-HPLC and SDS-PAGE data can be used to evaluate the stability of the pool.

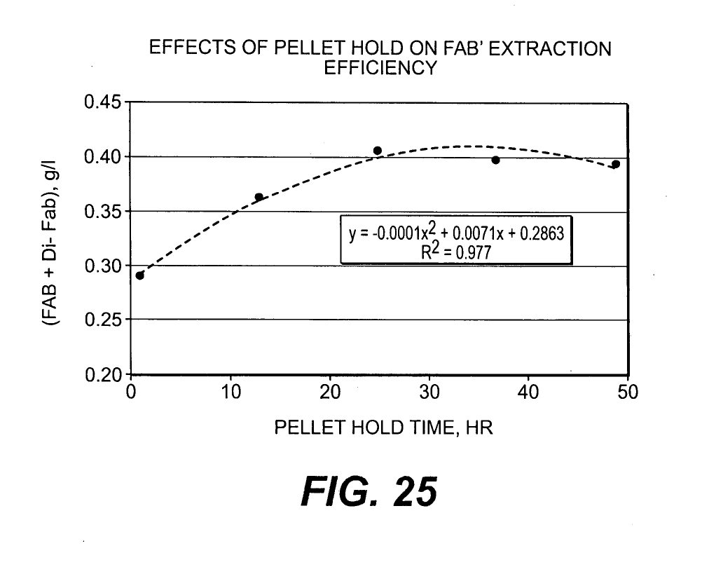

Parameters affecting the extraction process include extraction temperature, extraction time, extraction solution ionic strength, and pH. It is now reported that holding the cell paste for a period of time prior to the extraction step will result in a significant increase in the amount of Fab′ extracted into solution. Table 1 shows the effect of hold time on Fab′ species (i.e., Fab′, F(ab′)₂, Fab′ adducts, and any other species that can be reduced to form Fab′) extraction efficiency. For example at least a 30% increase in extraction yield was achieved with holding the cell paste at about 20° C. for about 30 hrs or longer prior to carrying out the typical extraction process described above. The data from Table 1 are plotted in FIG. 25.

TABLE 1
Effect of Pellet Hold Time on Fab'
Extraction Efficiency

| | | Relative |
|---|---|---|
| Pellet Hold | Fab' Species | Amount of |
| Time at 20° C., | Concentration in | Extracted |
| hr | Supernatant (mg/ml) | Fab' |
| 4 | 0.20 | 100% |
| 16 | 0.24 | 119% |
| 28 | 0.26 | 131% |
| 40 | 0.27 | 136% |
| 52 | 0.26 | 130% |

In addition to the length of hold time, the temperature at which the pellet is held can have an effect on the extraction yield. Table 2 shows the results obtained at 4, 20 and 37° C. holding temperature. As can be seen from the data, extraction yield increased as a function of hold temperature.

TABLE 2
Effect of hold time and temperature on Fab'
extraction efficiency

| | Fab' Species Concentration | | | |
|---|---|---|---|---|
| | in Supernatant (mg/ml) | | | |
| | | | | 1.5 L |
| Pellet Hold | 0.1 L Extraction | | | Extraction |
| Time, hr | 4° C. Hold | 20° C. Hold | 37° C. Hold | 20° C. Hold |
| 1 | | 0.29 | | 0.28 |
| 13 | | 0.36 | | |
| 25 | | 0.41 | | |
| 37 | | 0.40 | | 0.41 |
| 49 | 0.31 | 0.40 | 0.43 | |

The effect on the Fab′ extraction efficiency of the amount of fermentation broth left in the cell paste during the hold is shown in Table 8.

TABLE 8
Effects of Broth Level in Cell Pellet
on Fab Extraction

| | | | | Extracted |
|---|---|---|---|---|
| | Pellet Treatment | | | Fab' + |
| | (20° C.) | | | DiFab' in |
| | Hold | % Fermentation | Extraction | Supernatant, |
| Sample | Time, hr | Wt | Vol, L | mg/ml |
| 40% Broth Cell | 2.5 | 40 | 0.1 | 0.24 |
| Paste - (small | 50.5 | 40 | 0.1 | 0.32 |
| volume) | | | | |
| ˜20% Broth | 2.5 | ˜20 | 0.1 | 0.26 |
| Cell Paste - | 50.5 | ˜20 | 0.1 | 0.35 |
| (small volume) | | | | |
| 40% Broth Cell | | | | |
| Paste - (larger | 38.5 | 40 | 1.5 | 0.32 |
| volume) | | | | |
| Whole Broth | 0 | 100 | 0.1 | 0.29 |
| Extraction | | | | |

The extraction can be performed at a variety of pH's. For example, Table 9 shows the effect of extraction pH on the amount of Fab′ extracted based on RP-HPLC analysis (over 40% increase based on Protein G assay). It is useful to perform the extraction at a pH of about 7 or higher, alternatively about 8 or higher, in another embodiment about 9 or higher, and in another embodiment about 9 to about 11. In one embodiment, protein degradation reactions such as deamidation will be more extensive at higher pH. A pH of about 8 to about 9 will be useful to obtain an increased yield without significant product degradation.

TABLE 9
Effects of pH on Fab' Extraction

| | |
|---|---|
| Extraction | Extracted |
| conditions | Fab' (mg/L) |

| | | |
|---|---|---|
| Cntrl pH | Time = 0 hr | 50 |
| 7.4 | (initial) | |
| extracted | 4 hr | 230 |
| for 4 hr at | extraction | |
| 60° C. | (60° C.) | |
| pH 8 | T = 0 | 90 |
| extracted | 4 hr | 250 |
| for 4 hr at | extraction | |
| 60° C. | (60° C.) | |
| pH 9 | T = 0 | 120 |
| extracted | 4 hr | 320 |
| for 4 hr at | extraction | |
| 60° C. | (60° C.) | |
| pH 10 | T = 0 | 170 |
| extracted | 4 hr | 380 |
| for 4 hr at | extraction | |
| 60° C. | (60° C.) | |

Studies were carried out to explore the effect of extracting Fab′ from the cell paste using predetermined amounts of Tris and EDTA in the buffer solution without additional pH adjustment. This approach will eliminate the unnecessary salt addition from pH adjustment, which will simplify buffer preparation and subsequent column separation. All buffer combinations were prepared with a 1 M stock solution of Tris and a 200 mM stock solution of EDTA. The results in Table 10 show that unadjusted buffers with pH above 9 gave higher extraction efficiency over the control with lower solution conductivity.

TABLE 10
Fab′ extraction using Tris-EDTA buffers
without pH adjustment

| | | Initial | Final | Final | Total |
|---|---|---|---|---|---|
| Buffer | Buffer | Extraction | Extraction | conductivity | Fab′ |
| Conc | pH | pH | pH | (mS/cm) | (mg/L) |
| 200 mM | 7.4 | 7.08 | 5.67 | 12.78 | 270 |
| Tris/ | 9.0 | 8.63 | 7.59 | 9.08 | 380 |
| 20 mM | 11 | 9.08 | 8.10 | 8.47 | 460 |
| EDTA | Unadjusted | 8.78 | 7.86 | 8.28 | 460 |
| | (pH | | | | |
| | 9.22) | | | | |
| 600 mM | 7.4 | 7.56 | 6.05 | 21.7 | 290 |
| Tris/ | 9.0 | 8.94 | 8.14 | 9.68 | 490 |
| 20 mM | 11 | 9.42 | 8.61 | 7.64 | 430 |
| EDTA | Unadjusted | 9.23 | 8.38 | 7.59 | 450 |
| | (pH | | | | |
| | 9.67) | | | | |

Extraction experiments were conducted with two main chaotropic reagents, urea and guanidine (using 1-mL fermentation pellets in standard extraction buffer of 10 mM EDTA, 100 mM Tris pH 7.5). Shown in Table 11, addition of urea resulted in increases in Fab′ extraction.

TABLE 11
Extraction of fermentation pellets
Extraction buffer: 100 mM Tris, 10 mM EDTA, pH 7.5;
extraction temperature = 60° C.

| | | | % of Total Fab′ Area (RP-HPLC) | | | | | Fab′ + |
|---|---|---|---|---|---|---|---|---|
| Extraction | Additive | Extraction | Light | Heavy | HC/LC | Fab′ | F(ab′)₂ | F(ab′)₂ |
| Solution | Concentration | Time, hr | Chain | Chain | Ratio | Species | Species | mg/mL |
| Fab′ | | | 2.4% | 5.0% | 2.1 | 77.2% | 15.4% | |
| Reference | | | | | | | | |
| Extraction | None | 4 | 9.1% | 8.3% | 0.9 | 82.6% | 0.0% | 0.31 |
| Buffer | | 8 | 7.3% | 6.3% | 0.9 | 86.4% | 0.0% | 0.30 |
| +Guanidine | 1M | 4 | 6.6% | 25.6% | 3.9 | 67.8% | 0.0% | 0.32 |
| | | 8 | 6.3% | 25.9% | 4.1 | 67.8% | 0.0% | 0.31 |
| | 3M | 4 | 6.7% | 49.4% | 7.4 | 43.9% | 0.0% | 0.33 |
| | | 8 | 8.3% | 49.7% | 6.0 | 42.0% | 0.0% | 0.28 |
| | 6M | 4 | 31.1% | 43.5% | 1.4 | 25.3% | 0.0% | 0.38 |
| | | 8 | 29.3% | 44.3% | 1.5 | 26.4% | 0.0% | 0.21 |
| +Urea | 1M | 4 | 5.6% | 16.3% | 2.9 | 78.1% | 0.0% | 0.36 |
| | | 8 | 6.7% | 7.7% | 1.1 | 85.6% | 0.0% | 0.35 |
| | 3M | 4 | 5.4% | 27.4% | 5.0 | 67.2% | 0.0% | 0.44 |
| | | 8 | 4.9% | 24.7% | 5.0 | 70.4% | 0.0% | 0.43 |
| | 6M | 4 | 13.9% | 36.7% | 2.6 | 49.4% | 0.0% | 0.37 |
| | | 8 | 15.8% | 35.0% | 2.2 | 49.2% | 0.0% | 0.35 |

A study was carried out to test the effects of Tris and EDTA concentrations on the Fab′ extraction yield. The results are shown in Tables 12 and 13. In this study, EDTA concentration was held at 20 mM with Tris level ranging from 200 to 600 mM and solution pH adjusted independently to cover a range of 7.4 to 11. Analysis of the obtained data indicates that both high Tris and high pH at moderate level of EDTA increase Fab′ extraction.

In the fermentation, samples at the 30-hour time after induction were extracted with pH 7.4 buffer to produce 250 mg/L Fab′. Other samples at the 30-hour time after induction were extracted with pH 9.0 buffer to produce 340 mg/L Fab′.

TABLE 12
Effects of Tris & EDTA Extraction:
All buffer combinations were prepared with a 1 M
stock solution of Tris and a 200 mM stock solution of EDTA

| | | Final | | Extracted |
|---|---|---|---|---|
| Tris | EDTA | buffer | Extraction | Fab′ |
| (mM) | (mM) | pH | pH | (mg/L) |
| 300 | 20 | 11.06 | 9.08 | 390 |
| 500 | 100 | 11.12 | 9.78 | 270 |
| 200 | 50 | 11.08 | 9.43 | 360 |
| 100 | 50 | 11.05 | 9.25 | 300 |
| 500 | 20 | 11.12 | 9.39 | 370 |
| 300 | 50 | 11.07 | 9.57 | 480 |
| 150 | 15 | 11.07 | 9.01 | 310 |
| 300 | 10 | 11.02 | 9.27 | 380 |
| 100 | 100 | 11.04 | 9.53 | 340 |
| 100 | 20 | 11.04 | 8.93 | 330 |
| 500 | 10 | 11.07 | 9.35 | 410 |
| 500 | 50 | 11.14 | 9.51 | 450 |
| 150 | 15 | 11.04 | 8.97 | 310 |
| 150 | 15 | 11.07 | 8.9 | 310 |
| 200 | 10 | 11.04 | 9.04 | 390 |
| 100 | 10 | 10.98 | 8.97 | 350 |
| 200 | 20 | 11.07 | 9.26 | 400 |
| 300 | 100 | 11.12 | 9.47 | 340 |
| 200 | 100 | 11.02 | 9.46 | 290 |
| 200 | 20 | 9.01 | 8.68 | 340 |

TABLE 13
Effects of Tris & pH on Fab′ extraction

| Tris | EDTA | | | Post | Total |
|---|---|---|---|---|---|
| Conc | Conc | Buffer | Extraction | Extraction | Fab′ |
| (mM) | (mM) | pH | pH | pH | g/L |
| 600 | 20 | 9 | 8.92 | 8.16 | 450 |
| 200 | 20 | 9 | 8.67 | 7.58 | 330 |
| 600 | 20 | 7.4 | 7.53 | 5.74 | 190 |
| 600 | 20 | 11 | 9.43 | 8.63 | 90? |
| 400 | 20 | 7.4 | 7.28 | 5.57 | 220 |
| 600 | 20 | 11 | 9.41 | 8.59 | 550 |
| 400 | 20 | 9 | 8.85 | 7.95 | 410 |
| 300 | 20 | 8.2 | 8.84 | 7.97 | 400 |
| 200 | 20 | 7.4 | 6.72 | 5.52 | 160 |
| 200 | 20 | 9 | 8.66 | 7.47 | 380 |
| 200 | 20 | 11 | 9.08 | 8.17 | 310 |
| 600 | 20 | 7.4 | 7.49 | 5.60 | 170 |
| 400 | 20 | 9 | 8.87 | 7.98 | 300 |
| 400 | 20 | 11 | 9.30 | 8.52 | 310 |
| 600 | 20 | 9 | 8.97 | 8.19 | 350 |
| 400 | 20 | 7.4 | 7.06 | 5.50 | 150 |
| 300 | 20 | 8.2 | 8.82 | 7.96 | 290 |
| 200 | 20 | 7.4 | 6.44 | 5.48 | 150 |
| 200 | 20 | 11 | 9.08 | 8.13 | 270 |
| 400 | 20 | 11 | 9.28 | 8.39 | 360 |

The effects of various detergents, nonionic as well as ionic, on Fab′ extraction are shown in Table 14. Most of the detergents tested at room temperature show significant increases in the amount of Fab′ extracted relative to heated extraction. Useful detergents for extraction of Fab′ include octoxynol detergents (e.g., octoxynol-1, octoxynol-3, octoxynol-5, octoxynol-8, octoxynol-9, octoxynol-13, and others for example sold under the tradename Triton); polysorbate detergents (e.g., polyethoxylated polysorbates such as Tween 20, Tween 21, Tween 40, Tween 61, Tween 81, Tween 85, and others); alkylsulfates (e.g., sodium dodecyl sulfate or “SDS”); alkoyl sarcosine detergents (e.g., laruoyl sarcosine, cocoyl sarcosine, or lauroyl sarcsoinate, and others for example sold under the tradename Sarkosyl); benzalkonium chloride; or others.

TABLE 14
Effects of detergents on Fab′ extraction

| | % of Total | | | % of Total Fab′ | | | |
|---|---|---|---|---|---|---|---|
| | Area (RP-HPLC) | | | Area (RP-HPLC) | | (Fab′ + DiFab) | |
| Sample ID17070- | Light | Heavy | HC/LC | Fab′ | Di-Fab′ | mg/mL | |
| 010 | Chain | Chain | Ratio | Species | Species | RP-HPLC | Protein G |
| Fab′ Reference | 2.5% | 4.8% | 2.0 | 77.2% | 15.5% | | |
| Heat Extraction: | 9.1% | 8.3% | 0.9 | 82.6% | 0.0% | 0.31 | |
| 100 mM Tris, 10 mM | | | | | | | |
| EDTA, pH 7.4, | | | | | | | |
| 60° C. for 4 hr | | | | | | | |
| Extraction | | | | | | | |
| Buffer: 100 mM | | | | | | | |
| Tris, 10 mM | | | | | | | |
| EDTA, pH 7.4, | | | | | | | |
| 22° C., overnight | | | | | | | |
| +1% SDS | 37.9% | 34.5% | 0.9 | 27.5% | 0.0% | 0.58 | 0.30 |
| +1% sarkosyl | 35.8% | 31.7% | 0.9 | 32.5% | 0.0% | 0.61 | 0.52 |
| +1% deoxycholic | 37.2% | 29.1% | 0.8 | 33.7% | 0.0% | 0.60 | 0.47 |
| acid | | | | | | | |
| +1% benzalkonium | 38.4% | 10.7% | 0.3 | 50.8% | 0.0% | 0.38 | 1.04 |
| Cl- | | | | | | | |
| +1% Tween 20 | 36.1% | 28.2% | 0.8 | 35.6% | 0.0% | 0.38 | 0.50 |
| +1% Tween 85 | 36.3% | 29.1% | 0.8 | 34.6% | 0.0% | 0.35 | 0.46 |
| +1% Triton X- | 36.8% | 28.8% | 0.8 | 34.4% | 0.0% | 0.44 | 0.40 |
| 114 | | | | | | | |
| +1% Triton X- | 36.6% | 25.9% | 0.7 | 37.6% | 0.0% | 0.40 | 0.43 |
| 100 | | | | | | | |
| Extraction | | | | | | | |
| Buffer: 100 mM | | | | | | | |
| Tris, 10 mM | | | | | | | |
| Na₂EDTA, pH 9, | | | | | | | |
| 22° C. overnight | | | | | | | |
| +1% SDS | 38.0% | 38.5% | 1.0 | 23.6% | 0.0% | 0.46 | 0.38 |
| +1% sarkosyl | 34.6% | 35.7% | 1.0 | 29.7% | 0.0% | 0.48 | 0.49 |
| +1% deoxycholic | 37.9% | 28.8% | 0.8 | 33.3% | 0.0% | 0.48 | 0.47 |
| acid | | | | | | | |
| +1% | 27.1% | 6.5% | 0.2 | 66.4% | 0.0% | 0.27 | 0.93 |
| benzalkonium Cl- | | | | | | | |
| +1% Tween 20 | 35.0% | 29.6% | 0.8 | 35.4% | 0.0% | 0.51 | 0.51 |
| +1% Tween 85 | 34.9% | 28.6% | 0.8 | 36.5% | 0.0% | 0.48 | 0.46 |
| +1% Triton X- | 34.9% | 28.6% | 0.8 | 36.5% | 0.0% | 0.48 | 0.44 |
| 114 | | | | | | | |
| +1% Triton X- | 42.6% | 27.6% | 0.6 | 29.7% | 0.0% | 0.43 | 0.46 |
| 100 | | | | | | | |

Without limiting the scope of the present invention to any particular mechanism(s), there may be several explanations for the present observations regarding pellet hold time. One is that the cellular synthesis is still going at the cell paste stage. Thus, holding the cells could allow more time for the synthesis process to occur, making more products. Another possible mechanism is that the extra holding time results either in breakdown of the cell outer membrane making it more permeable, or results in additional transport of Fab′ from the cytoplasm into the periplasmic space where it can be extracted. Another possible mechanism is that the additional hold time may also allow the process of refold and assembly of light chains and heavy chains in the periplasm into Fab′ to carry out to greater completion.

### EXAMPLE 2a.

A typical extraction and sampling procedure is as follows.

Pellet Preparation:

Centrifuge four 500 mL centrifuge bottles (3000 RPM, 15 minutes), each containing 300 grams of production fermentation broth (from step 3 of FIG. 23). Decant supernatant from all four bottles to 40% original weight (120 g each). Combine three of the four bottles for extraction usage (total pellet weight 360 gm) into 500 ml. Aliquot the gently stirred pellet slurry into eight 250 ml flasks, 40 g in each flask. Cover the flasks with Parafilm. Randomize the flasks before the next step to minimize systematic error due to the order of pellet addition (e.g. solid settling out skewing the results towards later samples). Mark the flasks #1 through 6, with three for #5: #5R (room temperature), #5H (hot), #5C. (cold). Store flasks #1 through 6 and #5R on the bench (temperature ˜20° C.), #5C. in the refrigerator (˜4° C.), #5H in the 37° C. incubator. Note: Flasks #1 through 5 can be used for extraction experiments, #6 is a spare if needed

Extraction Experiments:

After Pellet hold time:

Add the following to flask containing 40 g pellet:
10 mL Distilled water
50 mL of Buffer (200 mM Tris, 20 mM EDTA, pH 7.5). Gently mix until mixture became homogeneous. Cover flask and placed in a shaker with temperature set at 60° C., 94 RPM.

### EXAMPLE 2a.

Alternative Cell Pellet Preparation.

An appropriate number of 500 mL centrifuge bottles, each containing 400 mL of harvest fermentation broth, are centrifuged at 3000 rpm for 15 minutes. Post centrifugation supernatant solutions from all bottles are decanted and saved together in a separate container. Cell pellets are then resuspended with supernatant to 40% of the original starting weight in each bottle (˜160 g each) and mixed well to form a slurry (to mimic the concentration effect which occurs during large scale centrifugation processing of harvest fermentation broth). All of the cell pellet slurries from each individual centrifuge bottle are then combined and mixed well to create a uniform mixture. A 40 mL aliquot of homogenous pellet slurry is transferred to each 250 ml flask. Ten mL of deionized water is then added to each 40 mL pellet slurry and mixed well. Additionally 50 mL of specified extraction buffer is added to cell slurry and mixed well. These 100 mL experiments are then incubated for 4 hrs at indicated temperature with 100 rpm mixing.

### EXAMPLE 3.

Gene Cloning and Expression of a Chimeric hTNF40 Antibody Molecule

RNA Preparation from hTNF40 Hybridoma Cells Total

RNA was prepared from 3×10⁷ hTNF40 hybridoma cells as described below. Cells were washed in physiological saline and dissolved in RNAzol (0.2 ml per 106 Cells). Chloroform (0.2 ml per 2 ml homogenate) was added, the mixture shaken vigorously for 15 seconds and then left on ice for 15 minutes. The resulting aqueous and organic phases were separated by centrifugation for 15 minutes in an Eppendorf centrifuge and RNA was precipitated from the aqueous phase-by the addition of an equal volume of isopropanol. After 15 minutes on ice, the RNA was pelleted by centrifugation, washed with 70% ethanol, dried and dissolved in sterile, RNAse free water. The yield of RNA was 400 micrograms.

PCR Cloning of hTNF40 Vh and VI

cDNA sequences coding for the variable domains of hTNF40 heavy and light chains were synthesised using reverse transcriptase to produce single stranded cDNA copies of the mRNA present in the total RNA, followed by Polymerase Chain Reaction (PCR) on the cDNAs with specific oligonucleotide primers.

a) cDNA Synthesis

cDNA was synthesized in a 20 microliter reaction volume containing the following reagents: 50 mM Tris-HCl. pH 8.3, 75 mM KCl, 10 mM dithiothreitol, 3 mM MgCl₂, 0.5 mM each deoxyribonucleoside triphosphate, 20 units RNAsin, 75 ng random hexanucleotide primer, 2 micrograms hTNF40 RNA and 200 units Moloney Murine Leukemia Virus reverse transcriptase. After incubation at 42° C. for 60 minutes, the reaction was terminated by heating at 95° C. for 5 minutes.

b) PCR

Aliquots of the cDNA were subjected to PCR using combinations of primers specific for the heavy and light chains. The nucleotide sequences of the 5′ primers for the heavy and right chains are shown in Tables 3 and 4 respectively. These sequences all contain, in order, a restriction site starting 7 nucleotides from their 5′ ends, the sequence GCCGCCACC (SEQ ID NO: 12), to allow optimal translation of the resulting mRNAs, an initiation codon and 20-30 nucleotides based on the leader peptide sequences of known mouse antibodies.

The 3′ primers are shown in Table 5. The light chain primer spans the J-C junction of the antibody and contains a restriction site for the enzyme Sp1I to facilitate cloning of the V1 PCR fragment. The heavy chain 3′ primers are a mixture designed to span the J-C junction of the antibody. The 3′ primer includes an ApaI restriction site to facilitate cloning. The 3′ region of the primers contains a mixed sequence based on those found in known mouse antibodies.

The combinations of primers described above enable the PCR products for Vh and V1 to be cloned directly into an appropriate expression vector (see below) to produce chimeric (mouse-human) heavy and light chains and for these genes to be expressed in mammalian cells to produce chimeric antibodies of the desired isotype.

Incubations (100 microliters) for the PCR were set up as follows. Each reaction contained 10 mM Tris-HCl pH 8.3, 1.5 mM MgCl₂, 50 mM KCl, 0.01% w/v gelatin, 0.25 mM each deoxyribonucleoside triphosphate, 10 pmoles 5′ primer′ mix (Table 6), 10 pmoles 3′ primer (CL12 (light chain) or R2155 (heavy chain) (Table 5)), 1 microliter cDNA and 1 unit Taq polymerase. Reactions were incubated at 95° C. for 5 minutes and then cycled through 94° C. for 1 minute, 55° C. for I minute and 72° C. for 1 minute. After 30 cycles, aliquots of each reaction were analysed by electrophoresis on an agarose gel. Light chain reactions containing 5′ primer mixes from light chain pools 1, 2 and 7 produced bands with sizes consistent with full length VI fragments while the reaction from heavy chain reaction pool 3 produced a fragment with a size expected of a Vh gene. The band produced by the light chain pool 1 primers was not followed up as previous results had shown that this band corresponds to a light chain pseudogene produced by the hybridoma cell. The band produced by the light chain pool 7 primers was weaker than the band from the pool 2 primers and therefore was not followed up. Only the band from light chain reaction pool 2, which was the strongest band, was followed up.

c) Molecular Cloning of the PCR Fragments

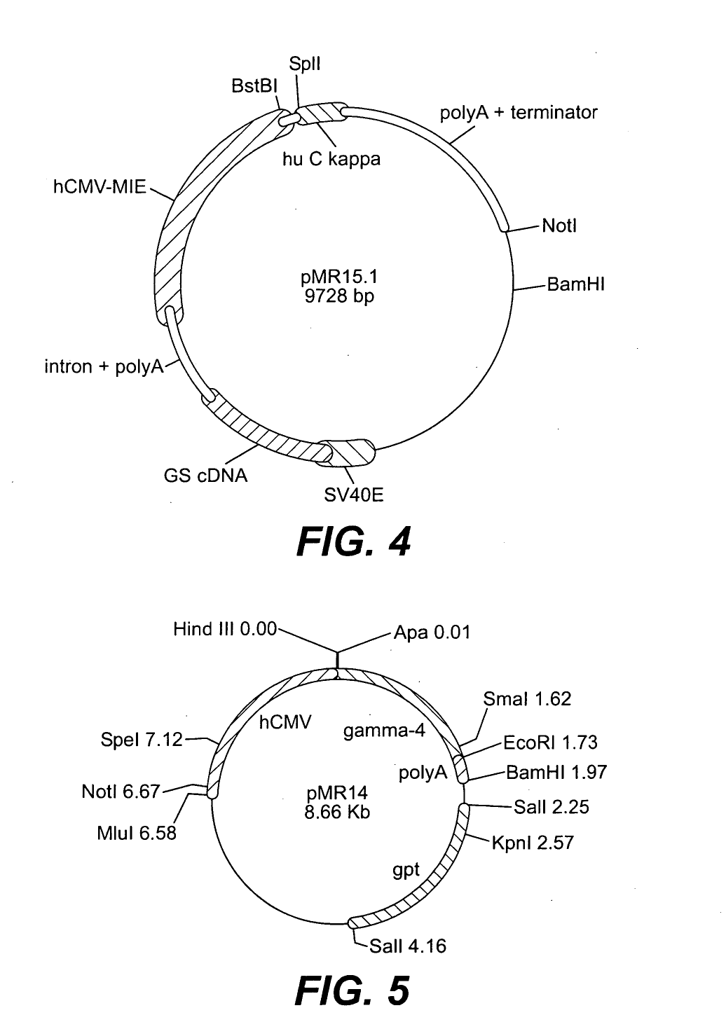

The DNA fragments produced in the light chain reaction pool 2 were digested with the enzymes BstBI and Sp1I, concentrated by ethanol precipitation, electrophoresed on a 1.4% agarose gel and DNA bands in the range of 400 base pairs recovered. These were cloned by ligation into the vector pMR15.1 (FIG. 4) that had been restricted with BstBI and Spll. After ligation, mixtures were transformed into E. coli LM 1035 and plasmids from the resulting bacterial colonies screened for inserts by digestion with BstBI and Sp1I. Representatives with inserts from each ligation were analyzed further by nucleotide sequencing.

In a similar manner, the DNA fragments produced in heavy chain reaction pool 3 were digested with HindIII and ApaI and cloned into the vector pMR14 (FIG. 5) that had been restricted with HindIII and ApaI. Again, representative plasmids containing inserts were analysed by nucleotide sequencing.

d) Nucleotide Sequence Analysis

Plasmid DNA from a number of isolates containing Vh inserts was sequenced using the primers R1053 (see Table 7) (which primes in the 3′ region of the HCMV promoter in pMR14) and R720 (see Table 7) (which primes in the 5′ region of human C-gamma 4 and allows sequencing through the DNA insert on pMR14). It was found that the nucleotide sequences of the Vh insert in a number of clones were identical, except for differences in the signal peptide and J regions. This indicated that the clones examined are independent isolates arising from the use of different primers from the mixture of oligonucleotides during the PCR stage. The determined nucleotide sequence and predicted amino acid sequence of the variable domain of the heavy chain of antibody hTNF40 (hTNF4OVh) are given in FIG. 7 (SEQ ID NO: 100).

To analyse the light chain clones, the sequence derived from priming with R1053 (see Table 7) and R684 (SEQ ID NO:62) (which primes in the 5′ region of human C-kappa and allows sequencing through the DNA insert on pMR15.1) was examined. The nucleotide sequence and predicted amino acid sequence of the VI genes arising from reactions in pool 2 were similarly analysed. Again it was found that the nucleotide sequences of the VI insert in a number of clones were identical, except for differences in the signal peptide and J regions, indicating that the clones examined were independent isolates arising from the use of different primers from the mixture of oligonucleotides used during the PCR stage. The determined nucleotide sequence and predicted amino acid sequence of the variable domain of the light chain of antibody hTNF40 (hTNF4OV1) are given in FIG. 6 (SEQ ID NO:99).

TABLE 3

Oligonucleotide primers for the 5′ region of
mouse heavy chains.

| | | |
|---|---|---|
| CH1: | 5′ATGAAATGCAGCTGGGTCAT (G,C) TTCTT3′ | (SEQ ID NO: 13) |
| CH2: | 5′ATGGGATGGAGCT (A,G) TATCAT (C,G) (C,T) TCTT3′ | (SEQ ID NO: 14) |
| CH3: | 5′ATGAAG (A,T) TGTGGTTAAACTGGGTTTT3′ | (SEQ ID NO: 15) |
| CH4: | 5′ATG (G,A) ACTTTGGG (T,C) TCAGCTTG (G,A) T3′ | (SEQ ID NO: 16) |
| CH5: | 5′ATGGACTCCAGGCTCAATTTAGTTTT3′ | (SEQ ID NO: 17) |
| CH6: | 5′ATGGCTGTC (C,T) T (G,A) G (G,C) GCT (G,A) CTCTTCTG3′ | (SEQ ID NO: 18) |
| CH7: | 5′ATGG (G,A) ATGGAGC (G,T) GG (G,A) TCTTT (A,C) TCTT3′ | (SEQ ID NO: 19) |
| CH8: | 5′ATGAGAGTGCTGATTCTTTTGTG3′ | (SEQ ID NO: 20) |
| CH9: | 5′ATGG (C,A) TTGGGTGTGGA (A,C) CTTGCTATT3′ | (SEQ ID NO: 21) |
| CH10: | 5′ATGGGCAGACTTACATTCTCATTCCT3′ | (SEQ ID NO: 22) |
| CH11: | 5′ATGGATTTTGGGCTGATTTTTTTTATTG3′ | (SEQ ID NO: 23) |
| CH12: | 5′ATGATGGTGTTAAGTCTTCTGTACCT3′ | (SEQ ID NO: 24) |

Each of the above primers has the sequence 5′GCGCGCAAGCTTGCCGCCACC3′ (SEQ ID NO: 25) added to its 5′ end.

TABLE 4

Oligonucleotide primers for the 5′ region of
mouse light chains.

| | | |
|---|---|---|
| CL1 | 5′ATGAAGTTGCCTGTTAGGCTGTTGGTGCT3′ | (SEQ ID NO: 26) |
| CL2 | 5′ATGGAG (T,A) CAGACACACTCCTG (T,C) TATGGGT3′ | (SEQ ID NO: 27) |
| CL3 | 5′ATGAGTGTGCTCACTCAGGTCCT3′ | (SEQ ID NO: 28) |
| CL4 | 5′ATGAGG (G,A) CCCCTGCTCAG (A,T) TT (C,T) TTGG3′ | (SEQ ID NO: 29) |
| CL5 | 5′ATGGATTT (T,A) CAGGTGCAGATT (T,A) TCAGCTT3′ | (SEQ ID NO: 30) |
| CL5A | 5′ATGGATTT (T,A) CA (A,G) GTGCAGATT (T,A) TCAGCTT3′ | (SEQ ID NO: 31) |
| CL6 | 5′ATGAGGT (T,G) C (T,C) (T,C) TG (T,C) T (G,C) AG (T,C) T (T,C) | (SEQ ID NO: 32) |
| | CTG (A,G) G3′ | |
| CL7 | 5′ATGGGC (T,A) TCAAGATGGAGTCACA3′ | (SEQ ID NO: 33) |
| CL8 | 5′ATGTGGGGA (T,C) CT (G,T) TTT (T,C) C (A,C) (A,C) TTTTTCAAT3′ | (SEQ ID NO: 34) |
| CL9 | 5′ATGGT (G,A) TCC (T,A) CA (G,C) CTCAGTTCCTT3′ | (SEQ ID NO: 35) |
| CL10 | 5′ATGTATATATGTTTGTTGTCTATTTC3′ | (SEQ ID NO: 36) |
| CLI I | 5′ATGGAAGCCCCAGCTCAGCTTCTCTT3′ | (SEQ ID NO: 37) |
| CL12A | 5′ATG (A,G) AGT (T,C) (A,T) CAGACCCAGGTCTT (T,C) (A,G) T3′ | (SEQ ID NO: 38) |
| CL12B: | 5′ATGGAGACACATTCTCAGGTCTTTGT3′ | (SEQ ID NO: 39) |
| CL13 | 5′ATGGATTCACAGGCCCAGGTTCTTAT3′ | (SEQ ID NO: 40) |
| CL14 | 5′ATGATGAGTCCTGCCCAGTTCCTGTT3′ | (SEQ ID NO: 41) |
| CL15 | 5′ATGAATTTGCCTGTTCATCTCTTGGTGCT3′ | (SEQ ID NO: 42) |
| CL16 | 5′ATGGATTTTCAATTGGTCCTCATCTCCTT3′ | (SEQ ID NO: 43) |
| CL17A: | 5′ATGAGGTGCCTA (A,G) CT (C,G) AGTTCCTG (A,G) G3′ | (SEQ ID NO: 44) |
| CL17B: | 5′ATGAAGTACTCTGCTCAGTTTCTAGG3′ | (SEQ ID NO: 45) |
| CL17C: | 5′ATGAGGCATTCTCTTCAATTCTTGGG3′ | (SEQ ID NO: 46) |

Each of the above primers has the sequence 5′GGACTGTTCGAAGCCGCCACC3′ (SEQ ID NO: 47) added to its 5′ end.

TABLE 5

Oligonucleotide primers for the 3′ ends of mouse
Vh and VI genes.

| | |
|---|---|
| Light chain (CL 12): | |
| 5′GGATACAGTTGGTGCAGCATCCGTACGTTT3′ | (SEQ ID NO: 48) |
| Heavy chain (R2155): | |
| 5′GCAGATGGGCCCTTCGTTGAGGCTG (A,C) (A,G) GAGAC (G,T,A) GTGA3′ | (SEQ ID NO: 49) |

TABLE 6
a) 5′ Primer mixtures for light chain PCR reactions pool 1
CL2.

pool 2 CL7.
pool 3 CL13.
pool 4 CL6.
pool 5 CL5A, CL9, CL17A.
pool 6 CL8.
pool 7 CL12A.
pool 8 CLI, CL3, CL4, CL5, CLIO, CLI 1, CUB, CL14,
CL15, CL16, CL17B, CL17C

b) 5′ Primer mixtures for heavy chain PCR reactions

pool 1: CH 1, CH2, CH3, CH4.
pool 2: CH5, CH6, CH7, CH8.
pool 3: CH9, CHIO, CHII, CH12.

TABLE 7
Primers used in nucleotide sequence analysis

| | | |
|---|---|---|
| R1053: | 5′GCTGACAGACTAACAGACTGTTCC3′ | (SEQ ID |
| | | NO: 50) |
| R720: | 5′GCTCTCGGAGGTGCTCCT3′ | (SEQ ID |
| | | NO: 51) |

Evaluation of Activities of Chimeric Genes

The activities of the chimeric genes were evaluated by expressing them in mammalian cells and purifying and quantitating the newly synthesised antibodies. The methodology for this is described below, followed by a description of the biochemical and cell based assays used for the biological characterisation of the antibodies.

a) Production of Chimeric hTNF40 Antibody Molecule.

Chimeric antibody for biological evaluation was produced by transient expression of the appropriate heavy and light chain pairs after co-transfection into Chinese Hamster Ovary (CHO) cells using calcium phosphate precipitation.

On the day prior to transfection, semi-confluent flasks of CHO-L761 cells were trypsinised, the cells counted and T75 flasks set up each with 10⁷ cells.

On the next day, the culture medium-was changed 3 hours before transfection. For transfection, the calcium phosphate precipitate was prepared by mixing 1.25 ml of 0.25 M CaCl₂ containing 50 micrograms of each of heavy and light chain expression vectors with 1.25 ml of 2×HBS (16.36 g NaCl, 11.0 g HEPES and 0.4 g Na₂HPO₄ in 1 liter water with the pH adjusted to 7.1 with NaOH) and adding immediately into the medium of the cells. After 3 hours at 37° C. in a CO₂ incubator, the medium and precipitate were removed and the cells shocked by the addition of 15 ml 15% glycerol in phosphate buffered saline (PBS) for 1 minute. The glycerol was removed, the cells washed once with PBS and incubated for 48 to 96 hours in 25 ml medium containing 10 mM sodium butyrate. Antibody could be purified from the culture medium by binding to and elution from protein A-Sepharose.

b) ELISA.

For the ELISA, Nunc ELISA plates were coated overnight at 4° C. with a F(ab′)₂ fragment of a polyclonal goat anti-human Fc fragment specific antibody (Jackson Immunoresearch, code 109-006-098) at 5 micrograms/mL in coating buffer (15 mM sodium carbonate, 35 mM sodium hydrogen carbonate, pH 6.9). Uncoated antibody was removed by washing 5 times with distilled water. Samples and purified standards to be quantitated were diluted to approximately 1 microgram/mL in conjugate buffer (0.1 M Tris-HCl, pH 7.0, 0.1 M NaCl, 0.2% v/v Tween 20, 0.2% w/v Harnmersten casein). The samples were titrated in the microtitre wells in 2-fold dilutions to give a final volume of 0.1 ml in each well and the plates incubated at room temperature for 1 hour with shaking. After the first incubation step the plates were washed 10 times with distilled water and then incubated for 1 hour as before with 0.1 ml of a mouse monoclonal anti-human kappa (clone GD12) peroxidase conjugated antibody (The Binding Site, code MP135) at a dilution of 1 in 700 in conjugate buffer. The plate was washed again and substrate solution (0.1 ml) added to each well. Substrate solution contained 150 microliters N,N,N,N-tetramethylbenzidine (10 mg/mL in DMSO), 150 microliters hydrogen peroxide (30% solution) in 10 ml 0.1 M sodium acetate/sodium citrate, pH 6.0. The plate was developed for 5-10 minutes until the absorbance at 630 nm was approximately 1.0 for the top standard. Absorbance at 630 nm was measured using a plate reader and the concentration of the sample determined by comparing the titration curves with those of the standard.

c) Determination of Affinity Constants by BiaCore Analysis.

The binding interaction between hTNF40 and human TNF was investigated using BIA technology. An affinity purified goat polyclonal antibody, directed against the constant region of hTNF40, was immobilised on the dextran polymer sensor chip surface using standard NHS/EDC chemistry. Relatively low levels (200-500 RU) of hTNF40 were captured to ensure mass transport effects were minimised. Human TNF at different concentrations was passed over the captured hTNF40 to allow assessment of the association kinetics. Following the injection of ligand, buffer was passed over the surface so that the dissociation could be measured. The association and dissociation rate constants for the interaction between solid phase hTNF40 and human TNF were calculated, and a KD value was derived.

### EXAMPLE 4.

CDR-Grafting of hTNF40

The molecular cloning of genes for the variable regions of the heavy and light chains of the hTNF40 antibody and their use to produce chimeric (mouse-human) hTNF40 antibodies has been described above. The nucleotide and amino acid sequences of the murine hTNF40 VI and Vh are shown in FIGS. 6 and 7 (SEQ ID NOS:99 and 100), respectively. This example describes the CDR-grafting of the hTNF40 antibody.

CDR-Grafting of hTNF40 Light Chain

Alignment of the framework regions of hTNF40 light chain with those of the four human light chain subgroups revealed that hTNF40 was most homologous to antibodies in human light chain subgroup 1. Consequently, for constructing the CDR-grafted light chain, the framework regions chosen corresponded to those of the human group 1 consensus sequence.

A comparison of the amino acid sequences of the framework regions of murine hTNF40 and the consensus human group 1 light chains is given in FIG. 1 and shows that there are 22 differences (underlined) between the two sequences. Analysis of the contribution that any of these framework differences might have on antigen binding identified two residues for investigation; these are at positions 46 and 60. Based on this analysis, two versions of the CDR-grafted light chain were constructed. In the first of these, hTNF40-gL1 (SEQ ID NO:8), residues 46 and 60 are derived from the hTNF40 light chain while in the second, hTNF40-gL2 (SEQ ID NO:9), all residues are human consensus except residue number 60 which is from the hTNF40 light chain.

Construction of CDR-Grafted Light Chain hTNF40-gL1.

The construction of hTNF40-gLI is given below in detail. The following overlapping oligonucleotides (P7982-P7986) were used in the Polymerase Chain Reactions (PCR) to assemble a truncated grafted light chain. The assembled fragment lacks the antibody leader sequence and the first 17 amino acids of framework 1.

| | |
|---|---|
| oligo 1 P7982: | |
| 5′GAATTCAGGGTCACCATCACTTGTAAAGCCAGTCAGAACGTAGGTACTAAC | (SEQ H) NO: 52) |
| GTAGCCTGGTATCAGCAAA3′ | |
| oligo 2 P7983: | |
| 5′ATAGAGGAAAGAGGCACTGTAGATGAGGGCTTTTGGGGCTTTACCTGGTTT | (SEQ ID NO: 53) |
| TTGCTGATACCAGGCTACGT3′ | |
| oligo 3 P7984: | |
| 5′TACAGTGCCTCTTTCCTCTATAGTGGTGTACCATACAGGTTCAGCGGATCCG | (SEQ ID NO: 54) |
| GTAGTGGTACTGATTTCAC3′ | |
| oligo 4 P7985 | |
| 5′GACAGTAATAAGTGGCGAAATCTTCTGGCTGGAGGCTACTGATCGTGAGGGT | (SEQ ID NO: 55) |
| GAAATCAGTACCACTACCG3′ | |
| oligo 5 P7986: | |
| 5′ATTTCGCCACTTATTACTGTCAACAGTATAACATCTACCCACTCACATTCGGT | (SEQ ID NO: 56) |
| CAGGGTACTAAAGTAGAAATCAAACGTACGGAATTC3′ | |
| Fwd P7981: | |
| 5′GAATTCAGGGTCACCATCACTTGTAAAGCC3′ | (SEQ ID NO: 57) |
| Bwd P7980 | |
| 5′GAATTCCGTACGTTTGATTTCTACTTTAGT3′. | (SEQ ID NO: 58) |

A PCR reaction, 100 microliters, was set up containing, 10 mM Tris-HCl pH 8.3, 1.5 mM MgCl₂, 50 mM KCl, 0.01% w/v gelatin, 0.25 mM each deoxyribonucleoside triphosphate, 2 pmoles of P7982, P7983, P7984, P7985, P7986, 10 pmoles of P7980, P7981 and 1 unit of Taq polymerase. Reactions were cycled through 94° C. for 1 minute, 55° C. for 1 minute and 72° C. for 1 minute. After 30 cycles, each reaction was analysed by electrophoresis on an agarose gel and the PCR fragment excised from the gel and recovered using a Mermaid Kit.

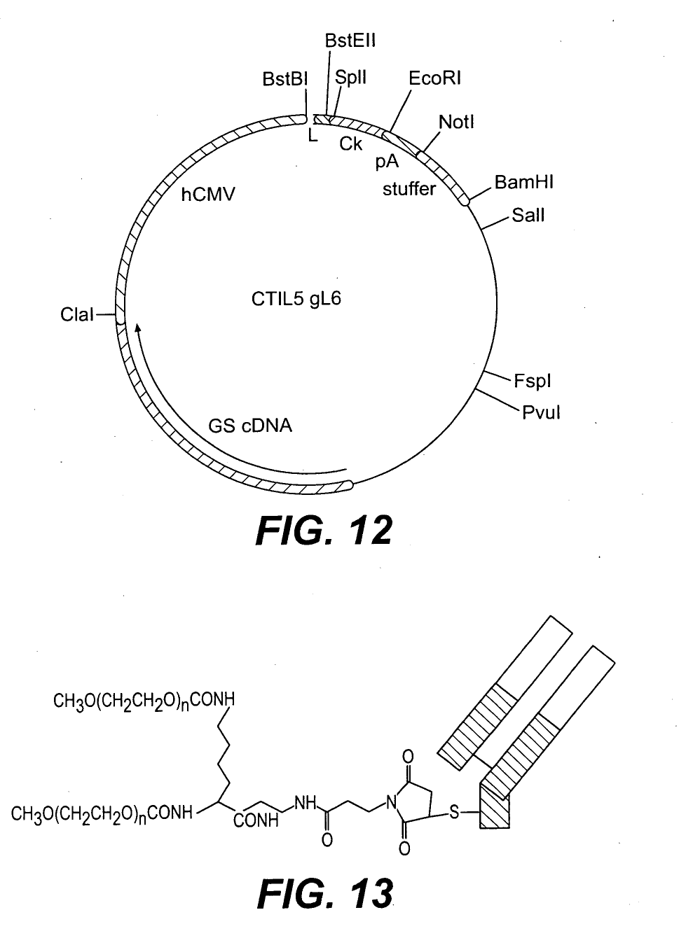

The recovered fragment was restricted with the enzymes BstEII and Sp1I in the appropriate buffer. The resulting product was finally electrophoresed on an agarose gel and the 270 base pair DNA fragment recovered from a gel slice and ligated into vector CTIL57gL6 (FIG. 12), that had previously been digested with the same enzymes. The above vector provides the missing antibody leader sequence and the first 17 amino acids of framework 1.

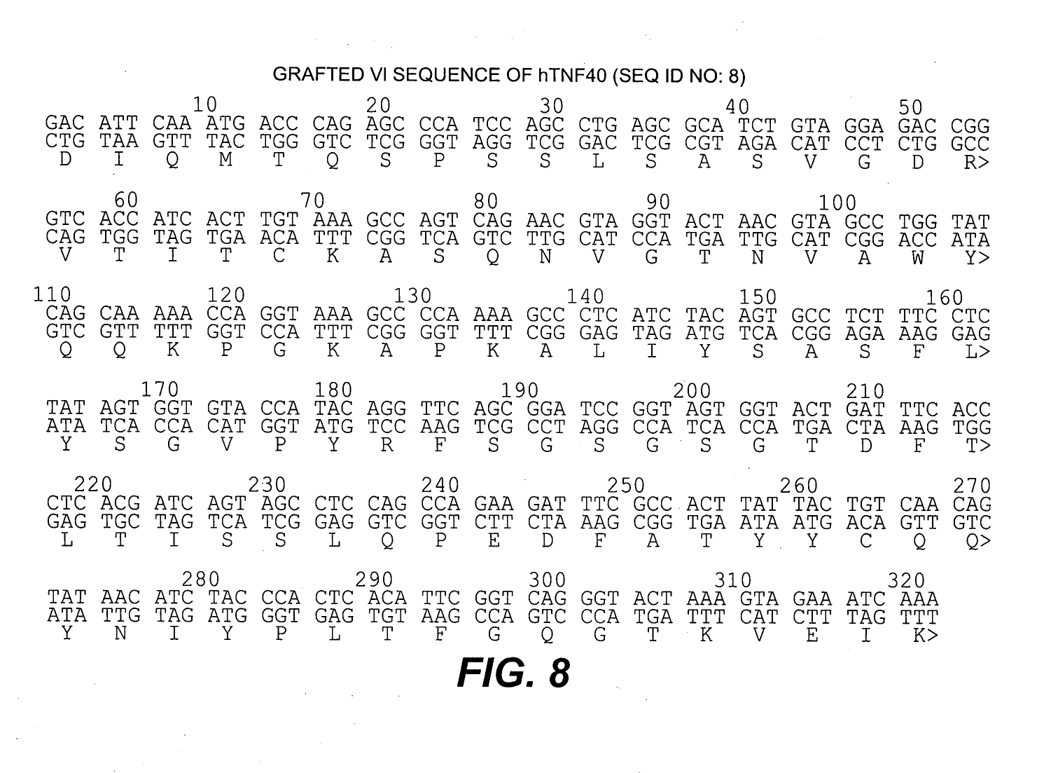

The ligation mixture was used to transform E. coli strain LM1035 and resulting colonies analysed by PCR, restriction enzyme digests and nucleotide sequencing. The nucleotide and amino acid sequence of the VI region of hTNF40-gLl is shown in FIG. 8 (SEQ ID NO:8).

Construction of CDR-Grafted Light Chain hTNF40-gL2.

hTNF40-gL2 (SEQ ID NO:9) was constructed using PCR. The following oligonucleotides were used to introduce the amino acid changes:

| | | |
|---|---|---|
| R1053: | 5′GCTGACAGACTAACAGACTGTTCC3′ | (SEQ ID NO: 59) 25 |
| R5350: | 5′TCTAGATGGCACACCATCTGCTAAGTTTGATGCAGCATAGAT | (SEQ ID NO: 60) |
| | CAGGAGCTTAGGAGC3′ | |
| R5349: | 5′GCAGATGGTGTGCCATCTAGATTCAGTGGCAGTGGATCA 30 | (SEQ ID NO: 61) |
| | GGCACAGACTTTACCCTAAC3′ | |
| R684: | 5′TTCAACTGCTCATCAGAT3′. | (SEQ ID NO: 62) |

Two reactions, each 20 microliters, were set up each containing 10 mM Tris-HCl pH 8.3, 1.5 mM MgCl₂, 50 mM KCl, 0.01% w/v gelatin, 0.25 mM each deoxyribonucleoside triphosphate, 0.1 micrograms hTNF40-gLl, 6 pmoles of R1053/R5350 or R5349/R684 and 0.25 units Taq polymerase. Reactions were cycled through 94° C. for 1 minute, 55° C. for 1 minute and 72° C. for I minute. After 30 cycles, each reaction was analyzed by electrophoresis on an agarose gel and the PCR fragments excised from the gel and recovered using a Mermaid Kit.

Aliquots of these were then subjected to a second round of PCR. The reaction, 100 microliters, contained 10 mM Tris-HCl pH 8.3, 1.5 MM MgCl₂, 50 mM KC1, 0.01% w/v gelatin, ⅕ of each of the PCR fragments from the first set of reactions, 30 pmoles of R1053 and R684 and 2.5 units Taq polymerase. Reaction temperatures were as above. After the PCR, the mixture was extracted with phenol/chloroform and then with chloroform and precipitated with ethanol. The ethanol precipitate was recovered by centrifugation, dissolved in the appropriate buffer and restricted with the enzymes BstEII and SplI. The resulting product was finally electrophoresed on an agarose gel and the 270 base pair DNA fragment recovered from a gel slice and ligated into the vector pMR15.1 (FIG. 4) that had previously been digested with the same enzymes.

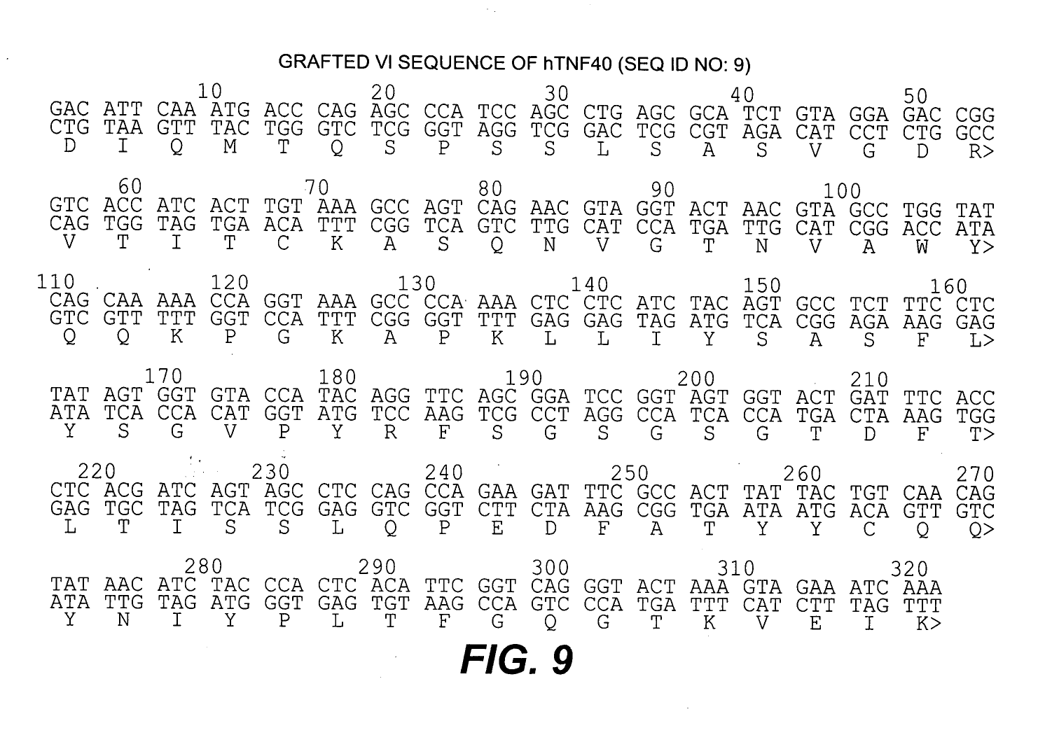

The ligation mixture was used to transform E. coli LM1035 and resulting colonies analyzed by PCR, restriction enzyme digests and nucleotide sequencing. The nucleotide and amino acid sequence of the VI region of hTNF40-gIL2 is shown in FIG. 9 (SEQ ID NO:9).

CDR-Grafting of hTNF40 Heavy Chain CDR-grafting of hTNF40 heavy chain was accomplished using the same strategy as described for the light chain. hTNF40 heavy chain was found to be most homologous to human heavy chains belonging to subgroup I and therefore the consensus sequence of the human subgroup 1 frameworks was chosen to accept the hTNF40 heavy chain CDRs.

To investigate the requirement of a homologous human framework to act as an acceptor framework for CDR grafting, a second framework, human group 3, was selected to humanize hTNF40 heavy chain.

A comparison of hTNF40 with the two different frameworks region is shown in FIG. 2 where it can be seen that hTNF40 differs from the human subgroup 1 consensus at 32 positions (underlined) and differs from the human subgroup 3 consensus at 40 positions (underlined). After analysis of the contribution that any of these might make to antigen binding, residues 28, 38, 46, 67, 69 and 71 were retained as donor in the CDR-grafted heavy chain ghlhTNF40.1, using the group 1 framework. Residues 27, 28, 30, 49, 49, 69, 71, 73, 76 and 78 were retained as donor in the CDR-grafted heavy chain, gh3hTNF40.4 using the group 3 framework. Residues 28, 69 and 71 were retained as donor in the CDR grafted heavy chain, ghlhTNF40.4 using the group I framework.

Construction of CDR-Grafted Heavy Chain ghlhTNF40.4

ghlhTNF40.4 (SEQ ID NO:10) was assembled by subjecting overlapping oligonucleotides to PCR in the presence of the appropriate primers. The following oligonucleotides were used in the PCR:

Group I graft

| | |
|---|---|
| oligo I P7989: | |
| 5′GAAGCACCAGGCTTCTTAACCTCTGCTCCTGACTGGACCAGCTGCACCTGA | (SEQ ID NO: 63) |
| G AGTGCACGAATTC3′ | |
| oligo 2 P7990: | |
| 5′GGTTAAGAAGCCTGGTGCTTCCGTCAAAGTTTCGTGTAAGGCCTCAGGCTA | (SEQ ID NO: 64) |
| C GTGTTCACAGACTATGGTA3′ | |
| oligo 3 P7991: | |
| 5′CCAACCCATCCATTTCAGGCCTTGTCCCGGGGCCTGCTTGACCCAATTCAT | (SEQ ID NO: 65) |
| AC CATAGTCTGTGAACACGT3′ | |
| oligo 4 P7995: | |
| 5′GGCCTGAAATGGATGGGTTGGATTAATACTTACATTGGAGAGCCTATTTAT | (SEQ ID NO: 66) |
| GT TGACGACTTCAAGGGCAGATTCACGTTC3′ | |
| oligo 5 P7992: | |
| 5′CCATGTATGCAGTGCGTTGTGGAGGTGTCTAGAGTGAACGTGAATCTGCCC | (SEQ ID NO: 67) |
| TT GAA3′ | |
| oligo 6 P7993: | |
| 5′CCACAAGCACTGCATACATGGAGCTGTCATCTCTGAGATCCGAGGACACCG | (SEQ ID NO: 68) |
| C AGTGTACTAT3′ | |
| oligo 7 P7994: | |
| 5′GAATTCGGTACCCTGGCCCCAGTAGTCCATGGCATAAGATCTGTATCCTCT | (SEQ ED NO: 69) |
| AG CACAATAGTACACTGCGGTGTCCTC3′ | |
| Fwd: P7988: | |
| 5′GAATTCGTGCACTCTCAGGTGCAGCTGGTC3′ | (SEQ ID NO: 70) |
| Bwd P7987: | |
| 5′GAATTCGGTACCCTGGCCCCAGTAGTCCAT3′ | (SEQ ED NO: 71) |

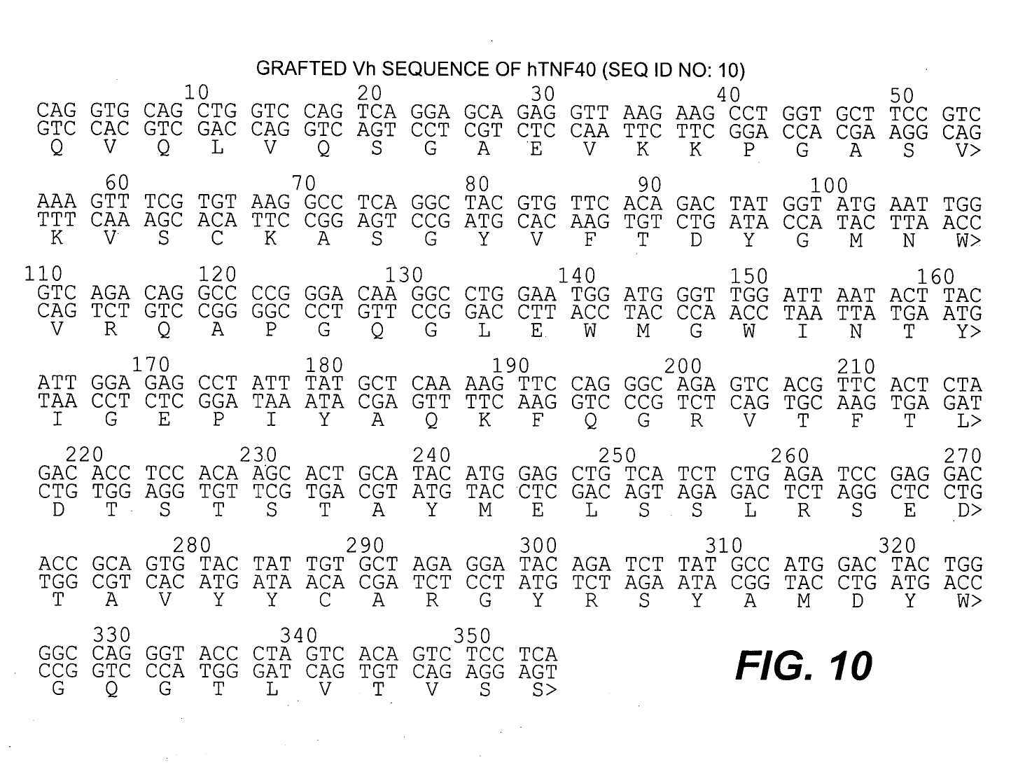

The assembly reaction, 100 microliters, contained 10 ml 4 Tris-HCl pH 8.3,1.5 MM MgCl₂, mM KCl, 0.01% w/v gelatin, 0.25 mM each deoxyribonucleoside triphosphate, 2 pmole of each of p7989, p7990, p7991, p7995, p7992, p7993 and p7994, 10 pmoles of each of p7988 and p7987 and 1 unit Taq polymerase. Reactions were cycled through 94° C. for 1 minute, 55° C. for 1 minute and 72° C. for 1 minute. After 30 cycles, the reaction was extracted with phenol/chloroform (1/1), then with chloroform and precipitated with ethanol. After centrifugation, the DNA was dissolved in the appropriate restriction buffer and digested with ApaLl and KpnI. The resulting fragment was isolated from an agarose gel and ligated into pMR14 (FIG. 5) that had previously been digested with the same enzymes. pMR14 contains the human gamma 4 heavy chain constant region when pMR14 is cleaved with ApaLl and KpnI, the cleaved vector is able to receive the digested DNA such that the 3′ end of the digested DNA joins in reading frame to the 5′ end of the sequence encoding the gamma 4 constant region. Therefore, the heavy chain expressed from this vector will be a gamma 4 isotype. The ligation mixture was used to transform E. coli LM1035 and resulting bacterial colonies screened by restriction digest and nucleotide sequence analysis. In this way, a plasmid was identified containing the correct sequence for ghlhTNF40.4 (FIG. 10) (SEQ ID NO: 10).

Construction of CDR-Grafted Heavy Chain gh3hTNF40.4

gh3hTNF40.4 (SEQ ID NO: 11) was assembled by subjecting overlapping oligonucleotides to PCR in the presence of the appropriate primers. The following 5′ oligonucleotides were used in the PCR:

Group 3 graft

| | |
|---|---|
| oligo, 1 P7999: | |
| 5′GATCCGCCAGGCTGCACGAGACCGCCTCCTGACTCGACCAGCTGAACCTCA | (SEQ ID NO: 72) |
| G AGTGCACGAATTC3′ | |
| oligo 2 P8000: | |
| 5′TCTCGTGCAGCCTGGCGGATCGCTGAGATTGTCCTGTGCTGCATCTGGTTA | (SEQ ID NO: 73) |
| CG 15 TCTTCACAGACTATGGAA3′ | |
| oligo 3 P8001 | |
| 5′CCAACCCATCCATTTCAGGCCCTTTCCCGGGGCCTGCTTAACCCAATTCATTC | (SEQ ID NO: 74) |
| CATAGTCTGTGAAGACGT3′ | |
| oligo 4 P7995: | |
| 5′GGCCTGAAATGGATGGGTTGGATTAATACTTACATTGGAGAGCCTATTTAT | (SEQ ID NO: 66) |
| GT TGACGACTTCAAGGGCAGATTCACGTTC3′ | |
| oligo 5 P7997: | |
| 5′GGAGGTATGCTGTTGACTTGGATGTGTCTAGAGAGAACGTGAATCTGCCCT | (SEQ ID NO: 75) |
| T GAA3′ | |
| oligo 6 P7998: | |
| 5′CCAAGTCAACAGCATACCTCCAAATGAATAGCCTGAGAGCAGAGGACACCG | (SEQ ID NO: 76) |
| C AGTGTACTAT3′ | |
| oligo 7 P7993: | |
| 5′GAATTCGGTACCCTGGCCCCAGTAGTCCATGGCATAAGATCTGTATCCTCT | (SEQ ID NO: 77) |
| AG CACAATAGTACACTGCGGTGTCCTC3′ | |
| Fwd P7996: | |
| 5′GAATTCGTGCACTCTGAGGTTCAGCTGGTC3′ | (SEQ ID NO: 79) |
| Bwd P7987: | |
| 5′GAATTCGGTACCCTGGCCCCAGTAGTCCAT3′ | (SEQ ID NO: 71) |

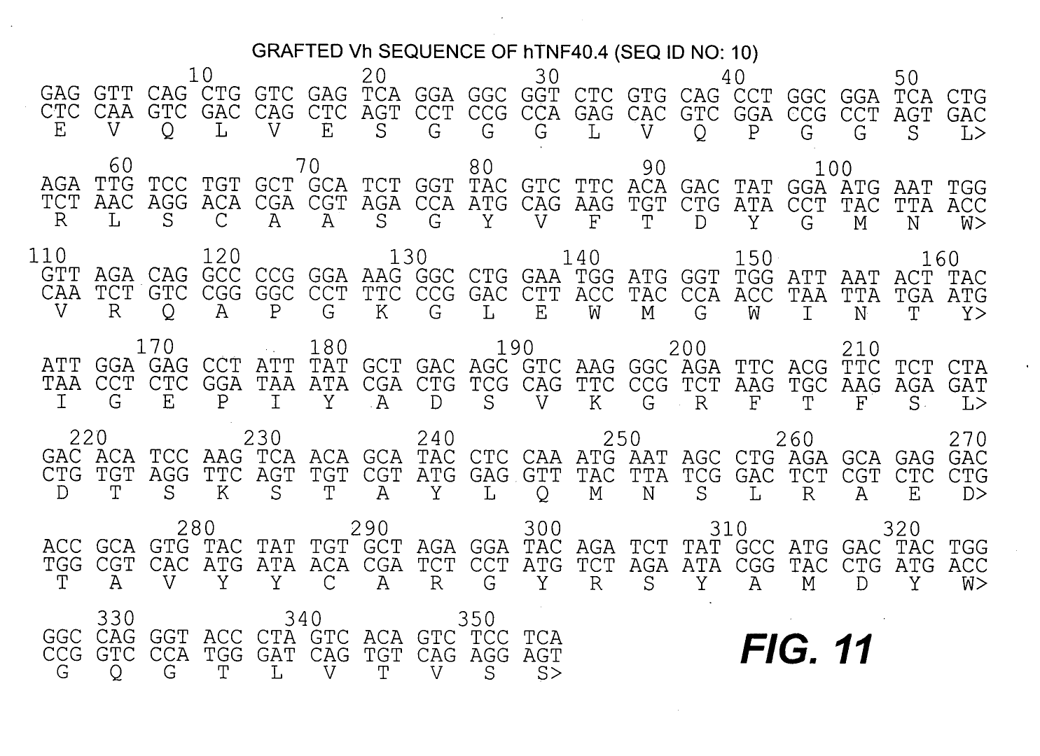

The assembly reaction, 100 microliters, contained 10 mM Tris-HCl pH 8.3, 1.5 MM MgCl₂, 50 mM KCl, 0.01% w/v gelatin, 0.25 mM each deoxyribonucleoside triphosphate, 2 pmole of each of p7999, p8000, p8001, p7995, p7997, p7998 and p7993, 10 pmoles of each of p7996 and p7987 and 1 unit Taq polymerase. Reactions were cycled through 94° C. for 1 minute, 55° C. for 1 minute and 72° C. for 1 minute. After 30 cycles, the reaction was extracted with phenol/chloroform (1/1), then with chloroform and precipitated with ethanol. After centrifugation, the DNA was dissolved in the appropriate restriction buffer and digested with ApaLI and KpnI. The resulting fragment was isolated from an agarose gel and ligated into pMR14 (FIG. 5) that had previously been digested with the same enzymes. pMR14 contained the human gamma 4 heavy chain constant region. When pMR14 is cleaved with ApaLl and KpnI, the cleaved vector is able to receive the digested DNA such that the 3′ end of the digested DNA joins in reading frame to the 5′ end of the sequence encoding the gamma 4 constant region. Therefore, the heavy chain expressed from this vector will be a gamma 4 isotype. The ligation mixture was used to transform E. coli LM1035 and resulting bacterial colonies screened by restriction digestion and nucleotide sequence analysis. In this way, a plasmid was identified containing the correct sequence for gh3hTNF40.4 (SEQ ID NO: 11) (FIG. 11).

Production of CDR-Grafted Modified Fab′ Fragment.

A CDR-grafted, modified Fab′ fragment, based on antibody hTNF40, was constructed using the E. coli vector pTTO-1. The variable regions of antibody hTNF40 are sub-cloned into this vector and the intergenic sequence optimized to create pTTO(CDP-870). The PTTO expression vector is designed to give rise to soluble, periplasmic accumulation of recombinant proteins in E. coli. The main features of this plasmid are:
(i) tetracycline resistance marker—antibiotic not inactivated by the product of resistance gene, hence selection for plasmid-containing cells is maintained;
(ii) low copy number—origin of replication derived from plasmid p15A, which is compatible with plasmids containing co1E1 derived replicons;
(iii) strong, inducible tac promoter for transcription of cloned gene(s);
(iv) lacI^{q} gene—gives constitutive expression of the lac repressor protein, maintaining the tac promoter in the repressed state until induction with IPTG/allolactose;
(v) OmpA signal sequence—gives periplasmic secretion of cloned gene(s); and
(vi) translational coupling of OmpA signal sequence to a short lacZ peptide, giving efficient initiation of translation.

The vector has been developed for expression of modified Fab′ fragments from a dicistronic message by the design of a method to select empirically the optimum intergenic sequence from a series of four purpose-built cassettes. The application of this in the construction of pTTO(CDP-870) is described.

Materials and Methods

DNA Techniques

Standard procedures were used for protocols including DNA restriction, agarose gel electrophoresis, ligation and transformation. Restriction enzymes and DNA modifying enzymes were obtained from New England Biolabs or Boehringer Mannheim, and were used according to the supplier's recommendations. DNA fragments were purified from agarose using the GeneClean protocol (BIO 101). Oligonucleotides were supplied by Oswel Oligonucleotide Service and were synthesized at the 40 nm scale. Plasmid DNA was isolated using Plasmid DNA Mini/Midi kits from Qiagen. PCR was performed using Perkin Elmer ‘Amplitaq’ as recommended. DNA sequencing was performed using the Applied Biosystems Taq cycle sequencing kit.

Shake-flask Induction

E. coli W3110 cultures were grown in L-broth supplemented with tetracycline (7.5 micrograms/mL). For inductions, fresh overnight cultures (grown at 30-C) were diluted to OD₆₀₀ Of 0.1 into 200 ml L-broth in a 2 L baffled flask and were grown at 30° C. in an orbital incubator. At OD₆₀₀ of 0.5, IPTG was added to 200 pM. Samples (normalized for OD) were taken at intervals.

Periplasmic Extraction

Culture samples were chilled on ice (5 minutes) then cells were harvested by centrifugation. Following resuspension in extraction buffer,(100 mM Tris.HCl, 10 MM EDTA, pH 7.4) samples were incubated overnight at 30-C, then clarified by centrifugation.

Assembly Assay

Modified Fab′ concentrations were determined by ELISA. Plates were coated at 4° C. overnight with anti-human Fd 6045 (2 micrograms/mL in coating buffer, physiological saline, 100 microliters per well). After washing, 100 pl of sample was loaded per well; purified A5B7 gamma-1 Fab′, initially at 2 micrograms/mL, was used as a standard. Samples were serially diluted 2-fold across the plate in sample conjugate buffer (per litre: 6.05 g trisaminomethane; 2.92 g NaCl; 0.1 Tween-20; 1 ml casein (0.2%)); plates were incubated for 1 hour at room temperature, with agitation. Plates were washed and dried, then 100 microliters of anti-human C-kappa (GD12)-peroxidase was added (diluted in sample conjugate buffer). Incubation was carried out at room temperature for 1 hour with agitation. Plates were washed and dried, then 100 microliters of substrate solution was added (10 ml sodium acetate/citrate solution (0.1 M pH 6); 100 microliters of H₂0₂ solution; 100 microliters tetramethylbenzidine solution (10 mg/ml in dimethylsulphoxide)). Absorbance at 630 nm was read 4-6 minutes after substrate addition.

Construction of Plasmid pTTO-1

(a) Replacement of the VTTQ9 Polylinker

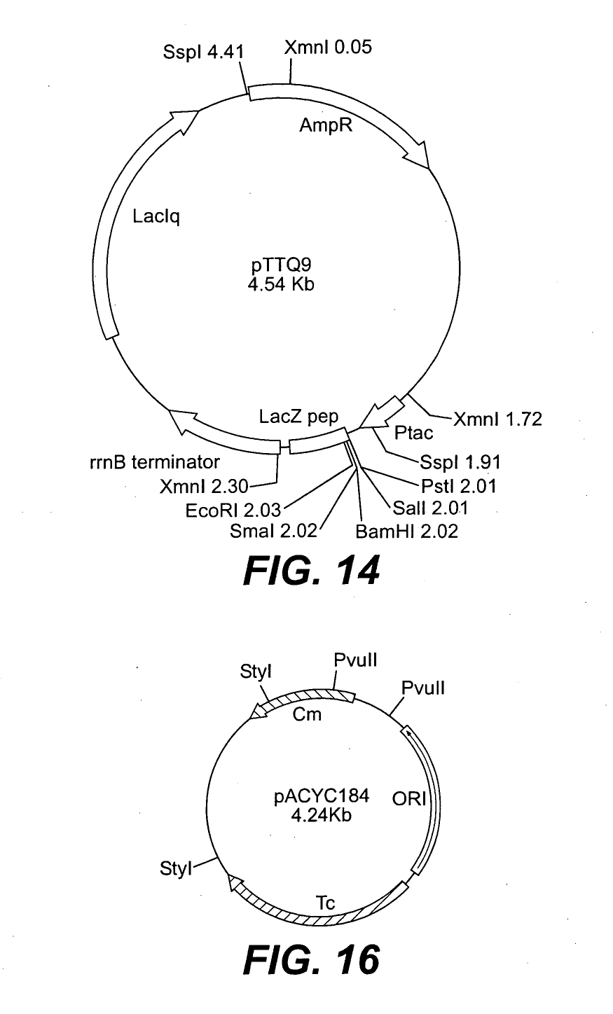

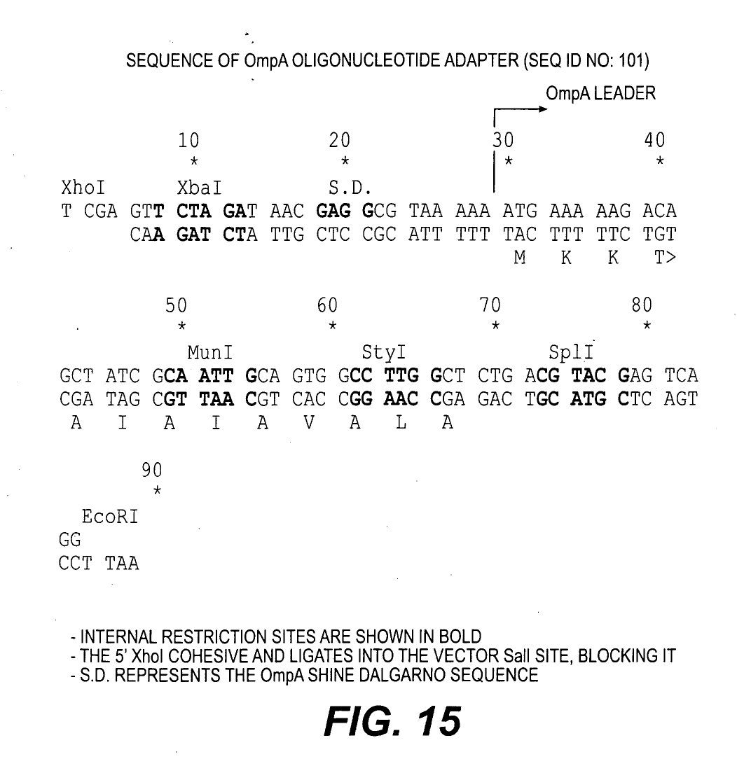

Plasmid pTTQ9 was obtained from Amersham and is shown in FIG. 14. An aliquot (2 micrograms) was digested with restriction enzymes SalI and EcoRI, the digest was run on a 1% agarose gel and the large DNA fragment (4520 bp) was purified. Two oligonucleotides were synthesized which, when annealed together, encode the OmpA polylinker region shown in FIG. 15. This sequence has cohesive ends which are compatible with the SalI and EcoRl ends generated by restriction of pTTQ9. By cloning this oligonucleotide, ‘cassette’ into the pTTQ9 vector, the SalI site is not regenerated, but the EcoRI site is maintained. The cassette encodes the first 13 amino acids of the signal sequence of the E. coli outer-membrane protein Omp-A, preceded by the Shine Dalgamo ribosome binding site of the OmpA gene. In addition restriction sites for enzymes XbaI, MunI, StyI and SplI are present. The MunI and StyI sites are within the coding region of 5 the OmpA signal sequence and are intended as the 5′cloning sites for insertion of genes. The two oligonucleotides which make up this cassette were annealed together by mixing at a concentration of 5 pmoles/microliter and heating in a waterbath to 95-C for 3 minutes, then slow cooling to room temperature. The annealed sequence was then ligated into the SalI/EcoRl cut pTTQ9. The resulting plasmid intermediate, termed pTQOmp, was verified by DNA sequencing.

(b) Fragment Preparation and Ligation

Plasmid pTTO-1 was constructed by ligating one DNA fragment from plasmid pACYC 184 to two fragments generated from pTQOmp. Plasmid pACYC 184 was obtained from New England Biolabs, and a restriction map is shown in FIG. 16. An aliquot (2 microgram) was digested to completion with restriction enzyme StyI, then treated with Mung Bean Nuclease; this treatment creates blunt ends by cutting back 5′ base overhangs. Following phenol extraction and ethanol precipitation, the DNA was restricted with enzyme PvuII, generating fragments of 2348, 1081, 412 and 403 bp. The 2348 bp fragment was purified after agarose gel electrophoresis. This fragment encodes the tetracycline resistance marker and the p15A origin of replication. The fragment was then treated with calf intestinal alkaline phosphatase to remove 5′ terminal phosphates, thereby preventing the self-ligation of this molecule.

An aliquot (2 micrograms) of plasmid pTQOmp was digested with enzymes SspI and EcoRI, and the 2350 bp fragment was purified from unwanted fragments of 2040 bp and 170 bp following agarose gel electrophoresis; this fragment encodes the transcriptional terminator region and the laCIq gene. Another aliquot (2 micrograms) of pTQOmp was digested with EcoRl and Xmnl, generating fragments of 2289, 1670, 350 and 250 bp. The 350 bp fragment, encoding the tac promoter, OmpA signal sequence and multicloning site, was gel purified.

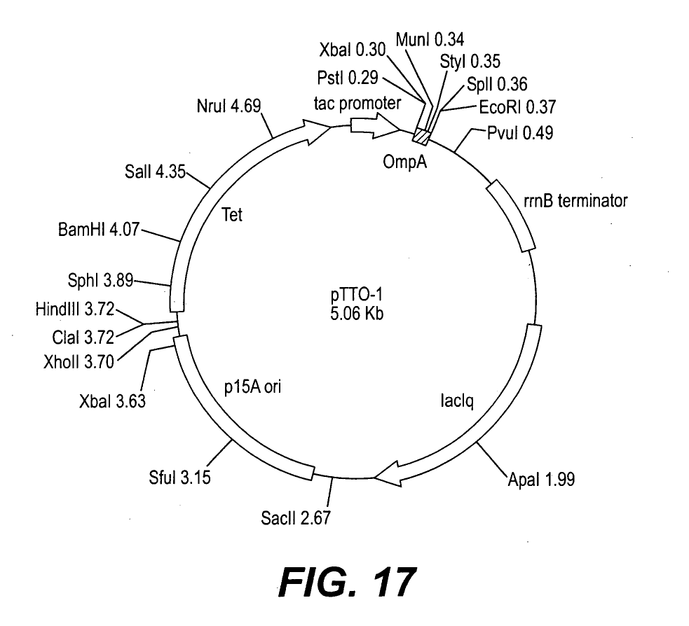

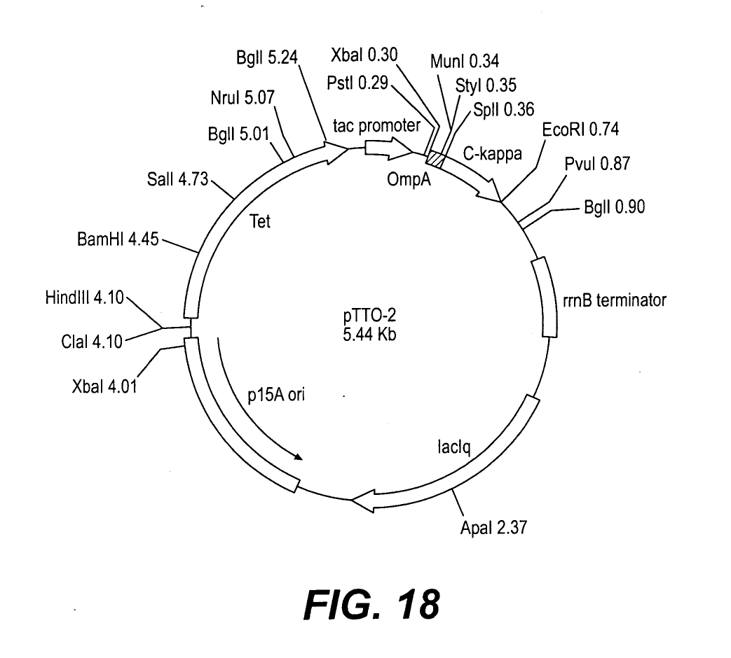

The three fragments were then ligated, using approximately equimolar amounts of each fragment, to generate the plasmid pTTO-1. All cloning junctions were verified by DNA sequencing. The restriction map of this plasmid is shown in FIG. 17. Plasmid pTTO-2 was then created by insertion of DNA encoding the human Ig light chain kappa constant domain. This was obtained as a Spl I-EcoRl restriction fragment from plasmid pHC132, and inserted into the corresponding sites in pTTO-1. Plasmid pTTO-2 is shown in FIG. 18.

Insertion of Humanized hTNF40 Variable Regions into PTTO-2

The variable light chain region hTNF4OgLl (SEQ ID NO:8) was obtained by PCR ‘rescue’ from the corresponding vector for mammalian cell expression pMR10.1. The OmpA leader sequence replaces the native Ig leader. The sequence of the PCR primers is shown below:

| | |
|---|---|
| 5′ primer: | |
| CGCGCGGCAATTGCAGTGGCCTTGGCTGGTTTCGCTACCGTAGCGCAAG | (SEQ ID NO: 79) |
| CTGACATTCAAATGACCCAGAGCCC | |
| 3′ primer: | |
| TTCAACTGCTCATCAGATGG | (SEQ ID NO: 80) |

Following PCR under standard conditions, the product was purified, digested with enzymes MunI and Sp1I then gel purified. The purified fragment was then inserted into the MunI/Sp1I sites of pTTO-2 to create the light chain intermediate pTTO(hTNF40L).

The variable heavy chain region of gh3hTNF40.4 was obtained in the same way from the vector pGamma-4. The sequence of the PCR primers is shown below:

| | |
|---|---|
| 5′ primer: | |
| GCTATCGCAATTGCAGTGGCGCTAGCTGGTTTCGCCACCGTGGCGCAAG | (SEQ ID NO: 81) |
| CTGAGGTTCAGCTGGTCGAGTCAGGAGGC | |
| 3′ primer: | |
| GCCTGAGTTCCACGACAC | (SEQ ID NO: 82) |

Following PCR the product was purified, digested with enzymes NheI and ApaI then sub-cloned into the vector pDNAbEng-Gl (FIG. 19). After verification by DNA sequencing, the heavy chain was restricted with enzyme EcoRl and sub- cloned into the EcoRI site of pTTO(hTNF40L) to create the E. coli expression plasmid pTTO(hTNF40).

Optimisation of Intergenic Sequence for Modified Fab′ Expression

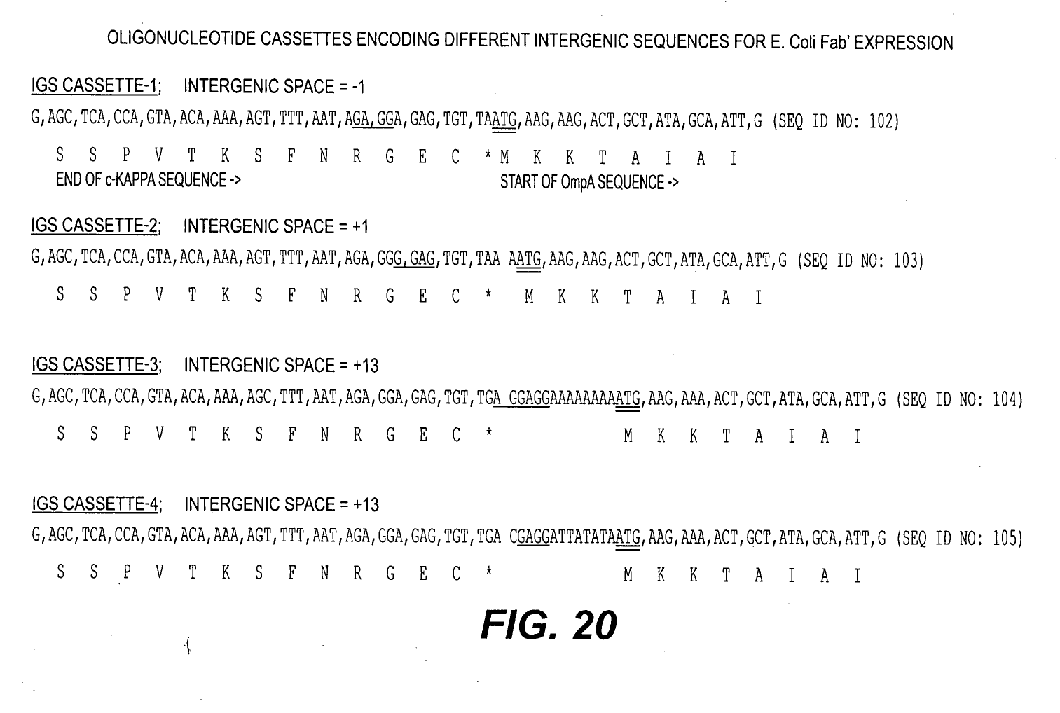

In the pTTO vector, modified Fab′ expression occurs from a dicistronic message encoding first light chain then heavy chain. The DNA sequence between the two genes (intergenic sequence, IGS) can influence the level of expression of the heavy chain by affecting the rate of translational initiation. For example, a short intergenic sequence may result in translational coupling between the light and heavy chains, in that the translating ribosome may not fully dissociate from the mRNA after completing light chain synthesis before initiating heavy chain synthesis. The ‘strength’ of any Shine Dalgamo (SD) ribosome binding site (homolgy to 16S rRNA) can also have an effect, as can the distance and sequence composition between the SD and the ATG start codon. The potential secondary structure of mRNA around the ATG can also have an effect; the ATG should be in a ‘loop’ and not constrained within a ‘stem’, while the reverse applies to the SD. Thus by modifying the composition and length of the IGS it is possible to modify the strength of translational initiation and therefore the level of heavy chain production. It is possible that an optimum rate of translational initiation needs to be achieved to maximise expression of the heavy chain of a given modified Fab. For example, with one modified Fab, a high level of expression may be tolerated, but for a different modified Fab′ with different amino acid sequence, a high level of expression might prove toxic, perhaps because of different efficiencies of secretion or folding. For this reason, a series of four intergenic sequences were designed (FIG. 20), permitting the empirical determination of the optimum IGS for the hTNF40-based modified Fab. IGS1 and IGS2 have very short intergenic sequences (−1 and +1 respectively) and might be expected to give closely coupled translation; the SD sequences (underlined) are subtly different. These two sequences will most likely confer a high level of translational initiation. IGS3 and IGS4 have a longer distance between start and stop codons (+13) and differ in their sequence composition; IGS3 has a ‘stronger’ SD sequence. All sequences were studied for secondary structure (using m/fold program) and ‘optimized’ as far as possible; however, with tight coupling of translation of the two chains the lack of ribosomal dissociation means that the mRNA may not be ‘naked’, preventing secondary structure formation.

Cloning of IGS Variants

The IGS cassettes shown in FIG. 20 have flanking SacI and MunI cloning sites. They were built by annealing complementary oligonucleotide pairs. A vector fragment was prepared by digesting pTTO(hTNF40) with SacI and NotI, and a heavy chain fragment was prepared by digesting pDNAbEngGl(hTNF40H) with MunI and NotI. Three-way ligations were then performed, using equimolar amounts of the two restriction fragments and approximately 0.05 pmoles of each annealed oligo cassette. This created the four expression plasmids pTTO(hTNF40 IGS-1), pTTO(hTNF40 IGS-2), pTTO(hTNF40 IGS-3), pTTO(hTNF40 IGS-4).

Shake Flask Expression Analysis

The four plasmids were transformed into E. coli strain W3110, along with the original expression construct, and then analysed for expression in shake flasks as described.

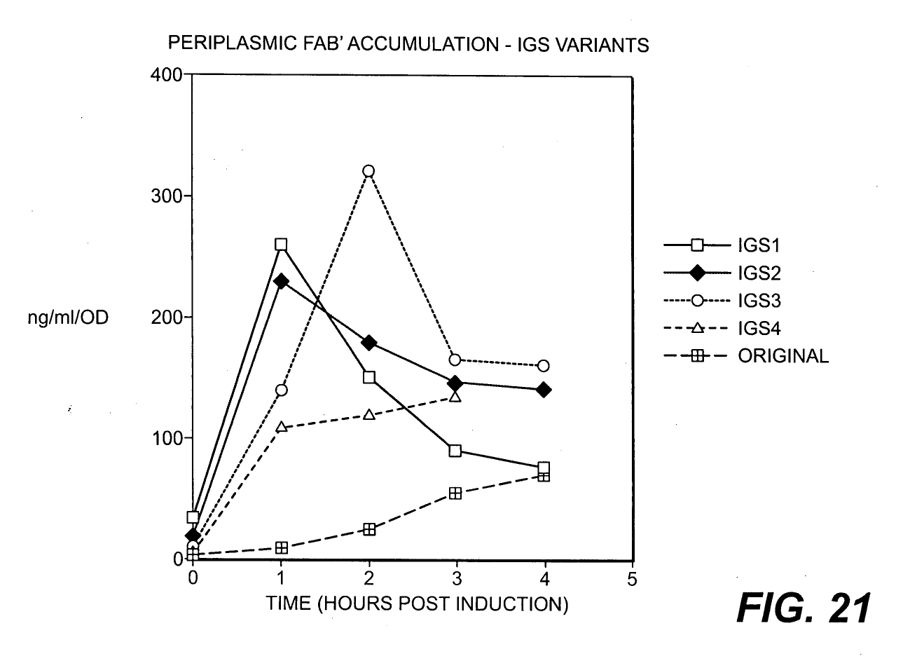

The results of a typical experiment are shown in FIG. 21. The different intergenic sequences confer different expression profiles. IGS1 and IGS2 accumulate periplasmic modified Fab′ rapidly with a peak at 1 hour post induction, after which the level recovered falls. The peak is greater and the fall sharper for IGS1. These results are consistent with a high level of synthesis, as expected for close translational coupling for these constructs.

IGS1 apparently confers a higher level of heavy chain expression than does IGS2. In this instance, it appears that this high level of expression is poorly tolerated, since periplasmic expression levels fall after the 1 hour peak. This is seen on the growth profile of the IGS1 culture (not shown), which peaks at hour post induction before falling, suggesting cell death and lysis. IGS3 accumulates modified Fab′ more slowly but peaks at 2 hours post induction with a higher peak value (325 ng/ml/OD), before levels fall. The growth of this culture continued to 3 hours post induction and reached a higher peak biomass (not shown). This is consistent with a lower level of heavy chain synthesis. IGS4 accumulates material at a slower rate still and fails to reach the high peak of productivity of the other three constructs. All IGS variants out-perform the original vector significantly. The hypothesis that the different IGS sequences confer different rates of translational initiation is supported by these experimental results. For, the hTNF40-based modified Fab′ it appears that a high rate of heavy chain translational initiation is poorly tolerated and is therefore not optimal. A slower rate, as conferred by IGS3, results in better growth characteristics and consequently a better yield accumulates over time.

Following comparison of productivity in the fermenter the IGS3 construct was selected as the highest yielding and was termed pTTO(CDP-870) (see FIG. 22).

The heavy chain encoded by the plasmid pTTO(CDP-870) has the sequence given in SEQ ID NO:115 and the light chain has the sequence given in SEQ ID NO:113.

Assays

Analytical Method for CDP-870 Fab′.

The analytical method used for CDP-870 Fab′ assay is reverse phase HPLC using a C4 Vydac column and a gradient of A (4.5 M Guanidine, 5 mM Phosphate, pH 6.5) and B(3M Guanidine, 50% IPA, 5 mM Sodium Phosphate).

Assay for Reduction of Fab′ Species.

Stachowicz, M. et.al., “Determination of total cysteamine in human serum by high-performance liquid chromatography with fluorescence detection” Journal of Pharmaceutical and Biomedical Analysis (1998), 17(4,5), 767-773.

Assay for Protein G.

The analytical method for Protein G is reverse phase HPLC using the conditions below:
Solutions and Buffer:
20 mM Sodium Phosphate, pH 7.2 (mobile phase A)
20 mM Sodium Phosphate, pH 2.5 (mobile phase B)
Column:
1 mL HiTrap Protein G HP
Cat. No. 170404-01
Method:
Run time: 6 min
Flow rate: 2 mL/min
Absorbance: 220 nm
Equilibrate the column with mobile phase A.
Inject 10 microliters of sample neat.
Elute with mobile phase B using a step elution.

The examples herein can be performed by substituting the generically or specifically described reactants and/or operating conditions of this invention for those used in the preceding examples.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

1. A method for the preparation of an antibody having a heavy chain and a light chain, wherein the method comprises:
fermenting a cell mixture comprising the cells and a supernatant solution, wherein the cells are capable of expressing the light chain and the heavy chain;
separating the cells from the supernatant solution to form a cell pellet;
allowing the cell pellet to stand for a hold time; and
extracting the cell pellet with an extracting solution,
thereby producing the TNF-alpha antibody fragment or a dimer or an adduct thereof.

2. The method of claim 1 wherein the antibody is a TNF-alpha antibody fragment or a dimer or an adduct thereof wherein the antibody or fragment has a 5′ part having the sequence given as SEQ ID NO: 115, and a light chain having the sequence given in SEQ ID NO: 113.

3. The method of claim 2 wherein the hold time is about 1 to about 100 hours.

4. The method of claim 3 wherein the hold time is about 5 to about 75 hours.

5. The method of claim 4 wherein the hold time is about 10 to about 50 hours.

6. The method of claim 5 wherein the hold time is about 20 to about 40 hours.

7. The method of claim 6 wherein the hold time is about 25 to about 35 hours.

8. The method of claims 1 or 2 wherein the cell pellet is allowed to stand for a hold time at a temperature in the range of about 5° C. to about 50 ° C.

9. The method of claim 8 wherein the temperature is about 10° C. to about 40° C.

10. The method of claim 9 wherein the temperature is about 15° C. to about 40° C.

11. The method of claim 10 wherein the temperature is about 20° C. to about 37° C.

12. The method of claims 1 or 2 wherein the extraction is performed at a pH greater than about 7.

13. The method of claim 12 wherein the extraction is performed at a pH greater than about 9.

14. The method of claim 13 wherein the extraction is performed at a pH of about 8 to about 9.


SEQUENCE LISTING (115 sequences)

SEQ ID NO: 1 - protein, 5 aa, Artificial Sequence
  Description of Artificial SequencehTNF40 CDRH1
DYGMN

SEQ ID NO: 2 - protein, 17 aa, Artificial Sequence
  Description of Artificial SequencehTNF40/human hybrid CDRH2
WINTYIGEPIYADSVKG

SEQ ID NO: 3 - protein, 9 aa, Artificial Sequence
  Description of Artificial SequencehTNF40 CDRH3
GYRSYAMDY

SEQ ID NO: 4 - protein, 11 aa, Artificial Sequence
  Description of Artificial SequencehTNF40 CDRL1
KASQNVGTNVA

SEQ ID NO: 5 - protein, 7 aa, Artificial Sequence
  Description of Artificial SequencehTNF40 CDRL2
SASFLYS

SEQ ID NO: 6 - protein, 9 aa, Artificial Sequence
  Description of Artificial SequencehTNF40 CDRL3
QQYNIYPLT

SEQ ID NO: 7 - protein, 17 aa, Artificial Sequence
  Description of Artificial SequencehTNF40 CDRH2
WINTYIGEPIYVDDFKG

SEQ ID NO: 8 - DNA, 321 bp, Artificial Sequence
  CDS (1)..(321)
  Description of Artificial Sequence hTNF40-gLl
gacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaagccctcatctacagtgcctctttcctctatagtggtgtaccatacaggttcagcggatccggtagtggtactgatttcaccctcacgatcagtagcctccagccagaagatttcgccacttattactgtcaacagtataacatctacccactcacattcggtcagggtactaaagtagaaatcaaa

SEQ ID NO: 9 - DNA, 321 bp, Artificial Sequence
  CDS (1)..(321): Description of Artificial Sequence hTNF40-gL2
gacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaactcctcatctacagtgcctctttcctctatagtggtgtaccatacaggttcagcggatccggtagtggtactgatttcaccctcacgatcagtagcctccagccagaagatttcgccacttattactgtcaacagtataacatctacccactcacattcggtcagggtactaaagtagaaatcaaa

SEQ ID NO: 10 - DNA, 354 bp, Artificial Sequence
  CDS (1)..(354)
  Description of Artificial SequenceghlhTNF40.4
caggtgcagctggtccagtcaggagcagaggttaagaagcctggtgcttccgtcaaagtttcgtgtaaggcctcaggctacgtgttcacagactatggtatgaattgggtcagacaggccccgggacaaggcctggaatggatgggttggattaatacttacattggagagcctatttatgctcaaaagttccagggcagagtcacgttcactctagacacctccacaagcactgcatacatggagctgtcatctctgagatccgaggacaccgcagtgtactattgtgctagaggatacagatcttatgccatggactactggggccagggtaccctagtcacagtctcctca

SEQ ID NO: 11 - DNA, 354 bp, Artificial Sequence
  CDS (1)..(354)
  Description of Artificial Sequence gh3hTNF40.4
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctca

SEQ ID NO: 12 - DNA, 9 bp, Artificial Sequence
  Description of Artificial Sequencepart of a primer sequence
gccgccacc

SEQ ID NO: 13 - DNA, 26 bp, Artificial Sequence
  Description of Artificial Sequence primer CHI
atgaaatgcagctgggtcatsttctt

SEQ ID NO: 14 - DNA, 26 bp, Artificial Sequence
  Description of Artificial Sequenceprimer CH2
atgggatggagctrtatcatsytctt

SEQ ID NO: 15 - DNA, 26 bp, Artificial Sequence
  Description of Artificial Sequenceprimer CH3
atgaagwtgtggttaaactgggtttt

SEQ ID NO: 16 - DNA, 23 bp, Artificial Sequence
  Description of Artificial Sequenceprimer CH4
atgractttgggytcagcttgrt

SEQ ID NO: 17 - DNA, 26 bp, Artificial Sequence
  Description of Artificial Sequenceprimer CH5
atggactccaggctcaatttagtttt

SEQ ID NO: 18 - DNA, 26 bp, Artificial Sequence
  Description of Artificial Sequenceprimer CH6
atggctgtcytrgsgctrctcttctg

SEQ ID NO: 19 - DNA, 26 bp, Artificial Sequence
  Description of Artificial Sequenceprimer CH7
atggratggagckggrtctttmtctt

SEQ ID NO: 20 - DNA, 23 bp, Artificial Sequence
  Description of Artificial Sequenceprimer CH8
atgagagtgctgattcttttgtg

SEQ ID NO: 21 - DNA, 26 bp, Artificial Sequence
  Description of Artificial Sequenceprimer CH9
atggmttgggtgtggamcttgctatt

SEQ ID NO: 22 - DNA, 26 bp, Artificial Sequence
  Description of Artificial Sequenceprimer CH10
atgggcagacttacattctcattcct

SEQ ID NO: 23 - DNA, 28 bp, Artificial Sequence
  Description of Artificial Sequenceprimer CH11
atggattttgggctgattttttttattg

SEQ ID NO: 24 - DNA, 26 bp, Artificial Sequence
  Description of Artificial Sequence primer CH12
atgatggtgttaagtcttctgtacct

SEQ ID NO: 25 - DNA, 21 bp, Artificial Sequence
  Description of Artificial Sequence primer 5′end
gcgcgcaagcttgccgccacc

SEQ ID NO: 26 - DNA, 29 bp, Artificial Sequence
  Description of Artificial Sequenceprimer CL1
atgaagttgcctgttaggctgttggtgct

SEQ ID NO: 27 - DNA, 29 bp, Artificial Sequence
  Description of Artificial Sequenceprimer CL2
atggagwcagacacactcctgytatgggt

SEQ ID NO: 28 - DNA, 23 bp, Artificial Sequence
  Description of Artificial Sequenceprimer CL3
atgagtgtgctcactcaggtcct

SEQ ID NO: 29 - DNA, 26 bp, Artificial Sequence
  Description of Artificial Sequenceprimer CL4
atgaggrcccctgctcagwttyttgg

SEQ ID NO: 30 - DNA, 29 bp, Artificial Sequence
  Description of Artificial Sequenceprimer CL5
atggatttwcaggtgcagattwtcagctt

SEQ ID NO: 31 - DNA, 29 bp, Artificial Sequence
  Description of Artificial Sequenceprimer CL5A
atggatttwcargtgcagattwtcagctt

SEQ ID NO: 32 - DNA, 26 bp, Artificial Sequence
  Description of Artificial Sequenceprimer CL6
atgaggtkcyytgytsagytyctgrg

SEQ ID NO: 33 - DNA, 23 bp, Artificial Sequence
  Description of Artificial Sequenceprimer CL7
atgggcwtcaagatggagtcaca

SEQ ID NO: 34 - DNA, 29 bp, Artificial Sequence
  Description of Artificial Sequenceprimer CLB
atgtggggayctktttycmmtttttcaat

SEQ ID NO: 35 - DNA, 24 bp, Artificial Sequence
  Description of Artificial Sequenceprimer CL9
atggtrtccwcasctcagttcctt

SEQ ID NO: 36 - DNA, 26 bp, Artificial Sequence
  Description of Artificial Sequenceprimer CL10
atgtatatatgtttgttgtctatttc

SEQ ID NO: 37 - DNA, 26 bp, Artificial Sequence
  Description of Artificial Sequence primer CL11
atggaagccccagctcagcttctctt

SEQ ID NO: 38 - DNA, 26 bp, Artificial Sequence
  Description of Artificial Sequenceprimer CL12A
atgragtywcagacccaggtcttyrt

SEQ ID NO: 39 - DNA, 26 bp, Artificial Sequence
  Description of Artificial Sequenceprimer CL12B
atggagacacattctcaggtctttgt

SEQ ID NO: 40 - DNA, 26 bp, Artificial Sequence
  Description of Artificial Sequenceprimer CL13
atggattcacaggcccaggttcttat

SEQ ID NO: 41 - DNA, 26 bp, Artificial Sequence
  Description of Artificial Sequenceprimer CL14
atgatgagtcctgcccagttcctgtt

SEQ ID NO: 42 - DNA, 29 bp, Artificial Sequence
  Description of Artificial Sequenceprimer CL15
atgaatttgcctgttcatctcttggtgct

SEQ ID NO: 43 - DNA, 29 bp, Artificial Sequence
  Description of Artificial Sequenceprimer CL16
atgaatttgcaattggtcctcatctcctt

SEQ ID NO: 44 - DNA, 26 bp, Artificial Sequence
  Description of Artificial Sequenceprimer CL17A
atgaggtgcctarctsagttcctarg

SEQ ID NO: 45 - DNA, 26 bp, Artificial Sequence
  Description of Artificial Sequenceprimer CL17B
atgaagtactctgctcagtttctagg

SEQ ID NO: 46 - DNA, 26 bp, Artificial Sequence
  Description of Artificial Sequence primer CL17C
atgaggcattctcttcaattcttggg

SEQ ID NO: 47 - DNA, 21 bp, Artificial Sequence
  Description of Artificial Sequenceprimer 5′ end
ggactgttcgaagccgccacc

SEQ ID NO: 48 - DNA, 30 bp, Artificial Sequence
  Description of Artificial Sequenceprimer CL12
ggatacagttggtgcagcatccgtacgttt

SEQ ID NO: 49 - DNA, 37 bp, Artificial Sequence
  Description of Artificial Sequenceprimer R2155
gcagatgggcccttcgttgaggctgmrgagacdgtga

SEQ ID NO: 50 - DNA, 24 bp, Artificial Sequence
  Description of Artificial Sequenceprimer R1053
gctgacagactaacagactgttcc

SEQ ID NO: 51 - DNA, 18 bp, Artificial Sequence
  Description of Artificial Sequenceprimer R720
gctctcggaggtgctcct

SEQ ID NO: 52 - DNA, 70 bp, Artificial Sequence
  Description of Artificial Sequence oligonucleotide P7982
gaattcagggtcaccatcacttgtaaagccagtcagaacgtaggtactaacgtagcctggtatcagcaaa

SEQ ID NO: 53 - DNA, 71 bp, Artificial Sequence
  Description of Artificial Sequence oligonucleotide P7983
atagaggaaagaggcactgtagatgagggcttttggggctttacctggtttttgctgataccaggctacgt

SEQ ID NO: 54 - DNA, 71 bp, Artificial Sequence
  Description of Artificial Sequence oligonucleotide P7984
tacagtgcctctttcctctatagtggtgtaccatacaggttcagcggatccggtagtggtactgatttcac

SEQ ID NO: 55 - DNA, 71 bp, Artificial Sequence
  Description of Artificial Sequence oligonucleotide P7985
gacagtaataagtggcgaaatcttctggctggaggctactgatcgtgagggtgaaatcagtaccactaccg

SEQ ID NO: 56 - DNA, 89 bp, Artificial Sequence
  Description of Artificial Sequence oligonucleotide P7986
atttcgccacttattactgtcaacagtataacatctacccactcacattcggtcagggtactaaagtagaaatcaaacgtacggaattc

SEQ ID NO: 57 - DNA, 30 bp, Artificial Sequence
  Description of Artificial Sequence oligonucleotide P7981
gaattcagggtcaccatcacttgtaaaacc

SEQ ID NO: 58 - DNA, 30 bp, Artificial Sequence
  Description of Artificial Sequence oligonucleotide P7980
gaattccgtacgtttgatttctactttagt

SEQ ID NO: 59 - DNA, 24 bp, Artificial Sequence
  Description of Artificial Sequence oligonucleotide R1053
gctgacagactaacagactgttcc

SEQ ID NO: 60 - DNA, 57 bp, Artificial Sequence
  Description of Artificial Sequence oligonucleotide R5350
tctagatggcacaccatctgctaagtttgatgcagcatagatcaggagcttaggagc

SEQ ID NO: 61 - DNA, 59 bp, Artificial Sequence
  Description of Artificial Sequence oligonucleotide R5349
gcagatggtgtgccatctagattcagtggcagtggatcaggcacagactttaccctaac

SEQ ID NO: 62 - DNA, 18 bp, Artificial Sequence
  Description of Artificial Sequence oligonucleotide R684
ttcaactgctcatcagat

SEQ ID NO: 63 - DNA, 65 bp, Artificial Sequence
  Description of Artificial Sequenceprimer P7989
gaagcaccaggcttcttaacctctgctcctgactggaccagctgcacctgagagtgcacgaattc

SEQ ID NO: 64 - DNA, 71 bp, Artificial Sequence
  Description of Artificial Sequenceprimer P7990
ggttaagaagcctggtgcttccgtcaaagtttcgtgtaaggcctcaggctacgtgttcacagactatggta

SEQ ID NO: 65 - DNA, 71 bp, Artificial Sequence
  Description of Artificial Sequenceprimer P7991
ccaacccatccatttcaggccttgtcccggggcctgcttgacccaattcataccatagtctgtgaacacgt

SEQ ID NO: 66 - DNA, 81 bp, Artificial Sequence
  Description of Artificial Sequenceprimer P7995
ggcctgaaatggatgggttggattaatacttacattggagagcctatttatgttgacgacttcaagggcagattcacgttc

SEQ ID NO: 67 - DNA, 56 bp, Artificial Sequence
  Description of Artificial Sequenceprimer P7992
ccatgtatgcagtgcgttgtggaggtgtctagagtgaacgtgaatctgcccttgaa

SEQ ID NO: 68 - DNA, 62 bp, Artificial Sequence
  Description of Artificial Sequenceprimer P7993
ccacaagcactgcatacatggagctgtcatctctgagatccgaggacaccgcagtgtactat

SEQ ID NO: 69 - DNA, 78 bp, Artificial Sequence
  Description of Artificial Sequenceprimer P7994
gaattcggtaccctggccccagtagtccatggcataagatctgtatcctctagcacaatagtacactgcggtgtcctc

SEQ ID NO: 70 - DNA, 30 bp, Artificial Sequence
  Description of Artificial Sequenceprimer P7988
gaattcgtgcactctcaggtgcagctggtc

SEQ ID NO: 71 - DNA, 30 bp, Artificial Sequence
  Description of Artificial Sequenceprimer P7987
gaattcggtaccctggccccagtagtccat

SEQ ID NO: 72 - DNA, 65 bp, Artificial Sequence
  Description of Artificial Sequenceprimer P7999
gatccgccaggctgcacgagaccgcctcctgactcgaccagctgaacctcagagtgcacgaattc

SEQ ID NO: 73 - DNA, 71 bp, Artificial Sequence
  Description of Artificial Sequenceprimer P8000
tctcgtgcagcctggcggatcgctgagattgtcctgtgctgcatctggttacgtcttcacagactatggaa

SEQ ID NO: 74 - DNA, 71 bp, Artificial Sequence
  Description of Artificial Sequenceprimer P8001
ccaacccatccatttcaggccctttcccggggcctgcttaacccaattcattccatagtctgtgaagacgt

SEQ ID NO: 75 - DNA, 55 bp, Artificial Sequence
  Description of Artificial Sequenceprimer P7997
ggaggtatgctgttgacttggatgtgtctagagagaacgtgaatctacccttgaa

SEQ ID NO: 76 - DNA, 62 bp, Artificial Sequence
  Description of Artificial Sequenceprimer P7998
ccaagtcaacagcatacctccaaatgaatagcctgagagcagaggacaccgcagtgtactat

SEQ ID NO: 77 - DNA, 78 bp, Artificial Sequence; identical to SEQ ID NO: 69
  Description of Artificial Sequenceprimer P7993

SEQ ID NO: 78 - DNA, 30 bp, Artificial Sequence
  Description of Artificial Sequenceprimer P7996
gaattcgtgcactctgaggttcagctggtc

SEQ ID NO: 79 - DNA, 74 bp, Artificial Sequence
  Description of Artificial Sequence 5′ primer
cgcgcggcaattgcagtggccttggctggtttcgctaccgtagcgcaagctgacattcaaatgacccagagccc

SEQ ID NO: 80 - DNA, 20 bp, Artificial Sequence
  Description of Artificial Sequence 3′ primer
ttcaactgctcatcagatgg

SEQ ID NO: 81 - DNA, 78 bp, Artificial Sequence
  Description of Artificial Sequence 5′ primer
gctatcgcaattgcagtggcgctagctggtttcgccaccgtggcgcaagctgaggttcagctggtcgagtcaggaggc

SEQ ID NO: 82 - DNA, 18 bp, Artificial Sequence
  Description of Artificial Sequence 3′ primer
gcctgagttccacgacac

SEQ ID NO: 83 - protein, 23 aa, Artificial Sequence
  Description of Artificial Sequencehuman group I consensus framework Ll
DIQMTQSPSSLSASVGDRVTITC

SEQ ID NO: 84 - protein, 23 aa, Artificial Sequence
  Description of Artificial SequencehTNF40 framework L1
DIVMTQSQKFMSTSVGDRVSVTC

SEQ ID NO: 85 - protein, 15 aa, Artificial Sequence
  Description of Artificial Sequencehuman group 1 consensus framework L2
WYQQKPGKAPKLLIY

SEQ ID NO: 86 - protein, 15 aa, Artificial Sequence
  Description of Artificial SequencehTNF40 framework L2
WYQQKPGQSPKALIY

SEQ ID NO: 87 - protein, 32 aa, Artificial Sequence
  Description of Artificial Sequencehuman group 1 consensus framework L3
GVPSRFSGSGSGTDFTLTISSLQPEDFATYYC

SEQ ID NO: 88 - protein, 32 aa, Artificial Sequence
  Description of Artificial SequencehTNF40 framework L3
GVPYRFTGSGSGTDFTLTISTVQSEDLAEYFC

SEQ ID NO: 89 - protein, 11 aa, Artificial Sequence
  Description of Artificial Sequencehuman group 1 consensus framework L4
FGQGTKVEIKR

SEQ ID NO: 90 - protein, 11 aa, Artificial Sequence
  Description of Artificial Sequence hTNF40 framework L4
FGAGTKLELKR

SEQ ID NO: 91 - protein, 30 aa, Artificial Sequence
  Description of Artificial Sequencehuman group 1 consensus framework H1
QVQLVQSGAEVKKPGASVKVSCKASGYTFT

SEQ ID NO: 92 - protein, 30 aa, Artificial Sequence
  Description of Artificial SequencehTNF40 framework H1
QIQLVQSGPELKKPGETVKISCKASGYVFT

SEQ ID NO: 93 - protein, 14 aa, Artificial Sequence
  Description of Artificial Sequencehuman group 1 consensus framework H2
WVRQAPGQGLEWMG

SEQ ID NO: 94 - protein, 14 aa, Artificial Sequence
  Description of Artificial SequencehTNF40 framework H2
WVKQAPGKAFKWMG

SEQ ID NO: 95 - protein, 32 aa, Artificial Sequence
  Description of Artificial Sequencehuman group 1 consensus framework H3
RVTITRDTSTSTAYMELSSLRSEDTAVYYCAR

SEQ ID NO: 96 - protein, 32 aa, Artificial Sequence
  Description of Artificial SequencehTNF40 framework H3
RFAFSLETSASTAFLQINNLKNEDTATYFCAR

SEQ ID NO: 97 - protein, 11 aa, Artificial Sequence
  Description of Artificial Sequencehuman group 1 consensus framework H4
WGQGTLVTVSS

SEQ ID NO: 98 - protein, 11 aa, Artificial Sequence
  Description of Artificial SequencehTNF40 framework H4
WGQGTTLTVSS

SEQ ID NO: 99 - DNA, 324 bp, murine
  CDS (1)..(324): mouse hTNF40 light chain variable domain
gacattgtgatgacccagtctcaaaaattcatgtccacatcagtaggagacagggtcagcgtcacctgcaaggccactcagaatgtgggtactaatgtagcctggtatcaacagaaaccaggacaatctcctaaagcactgatttactcggcatccttcctatatagtggagtcccttatcgcttcacaggcagtggatctgggacagatttcactctcaccatcagcactgtgcagtctgaagacttggcagagtatttctgtcagcaatataacatctatcctctcacgttcggtgctgggaccaagctggagctgaaacgt

SEQ ID NO: 100 - DNA, 354 bp, murine
  CDS (1)..(354): mouse hTNF40 heavy chain variable domain
cagatccagttggtgcagtctggacctgagctaaagaagcctggagagacagtcaagatctcctgcaaggcttctggatatgttttcacagactatggaatgaattgggtgaagcaggctccaggaaaggctttcaagtggatgggctggataaacacctacattggagagccaatatatgttgatgacttcaagggacgatttgccttctctttggaaacctctgccagcactgcctttttgcagatcaacaacctcaaaaatgaggacacggctacatatttctgtgcaagaggttaccggtcctatgctatggactactggggtcaaggaacctcagtcaccgtctcttca

SEQ ID NO: 101 - DNA, 84 bp, Artificial Sequence
  CDS (29)..(67): Description of Artificial SequenceOmpA oligonucleotide adaptor
tcgagttctagataacgaggcgtaaaaaatgaaaaagacagctatcacaattgcagtggccttggctctgacgtacgagtcagg

SEQ ID NO: 102 - DNA, 67 bp, Artificial Sequence
  CDS (2)..(40)
  CDS (43)..(66)
  Description of Artificial SequenceIGS cassette-1
gagctcaccagtaacaaaaagttttaatagaggagagtgttaatgaagaagactgctatagcaattg

SEQ ID NO: 103 - DNA, 69 bp, Artificial Sequence
  CDS (2)..(43)
  CDS (45)..(68)
  Description of Artificial SequenceIGS cassette-2
gagctcaccagtaacaaaaagttttaatagaggggagtgttaaaatgaagaagactgctatagcaattg

SEQ ID NO: 104 - DNA, 81 bp, Artificial Sequence
  CDS (2)..(43)
  CDS (57)..(80)
  Description of Artificial SequenceIGS cassette-3
gagctcaccagtaacaaaaagctttaatagaggagagtgttgaggaggaaaaaaaaatgaagaaaactgctatagcaattg

SEQ ID NO: 105 - DNA, 81 bp, Artificial Sequence
  CDS (2)..(43)
  CDS (57)..(80)
  Description of Artificial SequenceIGS cassette-4
gagctcaccagtaacaaaaagttttaatagaggagagtgttgacgaggattatataatgaagaaaactgctatagcaattg

SEQ ID NO: 106 - protein, 30 aa, Artificial Sequence
  Description of Artificial Sequencehuman group 3 consensus framework H1
EVQLVESGGGLVQPGGSLRLSCAASGFTFS

SEQ ID NO: 107 - protein, 14 aa, Artificial Sequence
  Description of Artificial Sequencehuman group 3 consensus framework H2
WVRQAPGKGLEWVS

SEQ ID NO: 108 - protein, 32 aa, Artificial Sequence
  Description of Artificial Sequencehuman group 3 consensus framework H3
RFTISRDNSKNTLYLQMNSLRAEDTAVYYCAR

SEQ ID NO: 109 - protein, 11 aa, Artificial Sequence
  Description of Artificial Sequencehuman group 3 consensus framework H4
WGQGTLVTVSS

SEQ ID NO: 110 - DNA, 648 bp, Artificial Sequence
  Grafted Heavy Chain for Fab
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctcagcttccaccaagggcccatcggtcttccccctggcaccctcctccaagagcacctctgggggcacagctgccttgggctgcctggtcaaggactacttccccgaaccggtgacggtgtcgtggaactcaggcgccctgaccagcggcgtgcacaccttctcggctgtcctacagtcctcaggactctactccctcagcagcgtggtgaccgtgccctccagcagcttgggcacccagacctacatctgcaacgtgaatcacaagcccagcaacaccaaggtcgacaagaaagtt

SEQ ID NO: 111 - protein, 216 aa, Artificial Sequence
  Grafted Heavy Chain for Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 112 - DNA, 642 bp, Artificial Sequence
  Grafted Light Chain for Fab and Modified Fab
gacattcaaatgacccagagcccatccagcctgagcgcatctgtaggagaccgggtcaccatcacttgtaaagccagtcagaacgtaggtactaacgtagcctggtatcagcaaaaaccaggtaaagccccaaaagccctcatctacagtgcctctttcctctatagtggtgtaccatacaggttcagcggatccggtagtggtactgatttcaccctcacgatcagtagcctccagccagaagatttcgccacttattactgtcaacagtataacatctacccactcacattcggtcagggtactaaagtagaaatcaaacgtacggtaacggccccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcaccagtaacaaaaagctttaatagaggagagtgt

SEQ ID NO: 113 - protein, 214 aa, Artificial Sequence
  Grafted Light Chain for Fab and Modified Fab
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - DNA, 687 bp, Artificial Sequence
  Grafted Heavy Chain for Modified Fab
gaggttcagctggtcgagtcaggaggcggtctcgtgcagcctggcggatcactgagattgtcctgtgctgcatctggttacgtcttcacagactatggaatgaattgggttagacaggccccgggaaagggcctggaatggatgggttggattaatacttacattggagagcctatttatgctgacagcgtcaagggcagattcacgttctctctagacacatccaagtcaacagcatacctccaaatgaatagcctgagagcagaggacaccgcagtgtactattgtgctagaggatacagatcttatgccatggactactggggccagggtaccctagtcacagtctcctcagcttccaccaagggcccatcggtcttccccctggcaccctcttccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccatcaacaccaaggtcgacaagaaagttgagcccaaatcttgtgacaaaactcacacatgcgccgcg

SEQ ID NO: 115 - protein, 229 aa, Artificial Sequence
  Grafted Heavy Chain for Modified Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA